(19) United States
(12) Patent Application Publication
(10) Pub. No.: US 2011/0182985 A1
(43) Pub. Date: Jul. 28, 2011

(54) Solid Pharmaceutical Composition with Enhancers and Methods of Preparing thereof

(76) Inventors: David C. Coughlan, Maynooth (IE); Thomas W. Leonard, Wilmington, NC (US); Bozena Adamczyk, Dublin (IE); Kieran Madigan, Dublin (IE); Edel O'Toole, Dublin (IE); Alan Cullen, Dublin (IE); Jason O'Hara, Dublin (IE)

(21) Appl. No.: 13/014,156

(22) Filed: Jan. 26, 2011

Related U.S. Application Data
(60) Provisional application No. 61/299,211, filed on Jan. 28, 2010.

(51) Int. Cl. A61K 31/727; A61K 47/26; A61K 31/663; A61K 9/48; A61K 9/00; A61P 19/10; A61P 7/02; A61P 19/02; A61P 35/00

(52) U.S. Cl. 424/452; 514/777; 514/56; 514/108; 424/400

(57) ABSTRACT

The present invention provides pharmaceutical compositions which are effective in providing therapeutically effective blood levels of a therapeutically active ingredient to a subject when administered to a gastrointestinal tract. In one aspect, the pharmaceutical compositions comprise a therapeutically effective amount of a therapeutically active ingredient; at least one water soluble enhancer, e.g., a medium chain fatty acid or a salt, ester, ether, or derivative of a medium chain fatty acid and has a carbon chain length of from about 4 to about 20 carbon atoms; and a saccharide.

### RELATED APPLICATION

This application claims the benefit under 35 U.S.C. §119(e) of U.S. Provisional Patent Application Ser. No. 61/299,211, filed Jan. 28, 2010, the disclosure of which is incorporated herein by reference in its entirety.

### FIELD OF THE INVENTION

The present invention generally relates to solid pharmaceutical compositions with absorption enhancers for oral administration and methods of preparing thereof. The compositions provide release characteristics for the therapeutically active ingredient and the enhancer that maximize the bioavailability of the therapeutically active ingredient.

### BACKGROUND OF THE INVENTION

Solid oral dosage forms, especially tablets, are the most common and preferred dosage forms for administering drugs or therapeutically active ingredients because they can be easily prepared and administered and have good stability. The preparation of tablets and some capsules requires the compositions to be compressible. Therapeutically active ingredients alone usually do not have the required characteristics of flow and compressibility necessary to prepare a solid oral dosage form. Therefore, additional excipients are usually added to impart suitable flow and compression characteristics to the composition.

For some therapeutically active ingredients, oral absorption from solid dosage forms may be limited in the gastro-intestinal tract, and thus an enhancer may be required to provide sufficient bioavailability of the active ingredient. The inclusion of excipients and enhancers in addition to the active ingredient may significantly increase the size of the oral dosage form such that it can not be orally administered, and/or may decrease the amount of the administered active ingredient in one dosage form, requiring administration of multiple dosages.

Therefore, there is a need in the industry for techniques and/or formulations that can provide an oral dosage form having reasonable characteristics that provide for sufficient absorption of the active ingredient.

### SUMMARY OF THE INVENTION

The present invention provides pharmaceutical compositions which are effective in providing therapeutically effective blood levels of a therapeutically active ingredient to a subject when administered to a gastrointestinal tract. In one aspect, the pharmaceutical compositions comprise a therapeutically effective amount of a therapeutically active ingredient; at least one water soluble enhancer; and a saccharide. The water soluble enhancer may be a medium chain fatty acid or a salt, ester, ether, or derivative of a medium chain fatty acid and has a carbon chain length of from about 4 to about 20 carbon atoms. In one embodiment, the therapeutically active ingredient and the enhancer are concurrently released at a substantially similar rate after the pharmaceutical composition enters the intestine of a subject. In another embodiment, the therapeutically active ingredient and the enhancer are released rapidly after the pharmaceutical composition enters the intestine of a subject. In a further embodiment, the therapeutically active ingredient is a bisphosphonate compound. In one embodiment, the saccharide is sorbitol. In another embodiment, the enhancer is sodium caprate.

Another aspect of the invention provides methods of providing the pharmaceutical compositions described herein for oral administration in one dosage unit with a patient acceptable size. In one aspect, the methods comprise directly compressing or dry granulating the enhancer without adding any moisture agent before preparing the dosage form.

A further aspect of the invention relates to methods for the treatment and/or prevention of a medical condition which are effective in providing therapeutically effective blood levels of a therapeutically active ingredient to a subject when administered to a gastrointestinal tract of the subject. The methods comprise administering orally to the subject a pharmaceutical composition described herein.

Objects of the present invention will be appreciated by those of skill in the art from a reading of the figures and the detailed description of the preferred embodiments which follow, such description being merely illustrative of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1-a graphically demonstrates the relationship between the percentage of total dose of sodium alendronate excreted in urine versus the amount of sodium caprate (C10) per administration. FIGS. 1-b and 1-c graphically show the dissolution profile of C10 for tablets containing different amount of C10, respectively. FIG. 1-b demonstrates dissolution profiles of C10 in phosphate buffer pH 6.8, which is expressed as % released C10 per tablet. FIG. 1-c demonstrates dissolution profiles of C10 in phosphate buffer pH 6.8, which is expressed as the amount of released C10 per tablet. FIG. 1-d graphically shows the correlation between in vivo performance [% sodium alendronate excreted in urine] and in vitro performance [Amount of sodium alendronate released at T=20 minutes in phosphate buffer pH 6.8 (USP Paddle Apparatus, 50 rpm, 37° C., 900 mL, 2 hrs in 0.1NHCl]. FIG. 1-e demonstrates the correlation between in vivo performance (% sodium alendronate excreted in urine) and in vitro performance (Amount of C10 released at T=20 minutes in phosphate buffer pH 6.8 (USP Paddle Apparatus, 50 rpm, 37° C., 900 mL, 2 hrs in 0.1N HCl).

FIG. 2 demonstrates the disintegration time of tablets including different excipients.

FIG. 3(a) graphically demonstrates the dissolution profile of zoledronic acid for tablets in EXP 1414. FIG. 3(b) graphically demonstrates the dissolution profile of zoledronic acid for tablets in EXP 1415.

FIG. 4(a) graphically demonstrates the dissolution profile of C10 for tablets in EXP 1414. FIG. 4(b) graphically demonstrates the dissolution profile of C10 for tablets in EXP 1415.

FIG. 5(a) graphically demonstrates the dissolution profile of zoledronic acid for tablets in EXP 1427 and 1428. FIG. 5(b) graphically demonstrates the first derivative plot of zoledronic acid for tablets in EXP 1427 and 1428.

FIG. 6(a) graphically demonstrates the dissolution profile of C10 for tablets in EXP 1427 and 1428. FIG. 6(b) graphically demonstrates the first derivative plot of C10 for tablets in EXP 1427 and 1428.

FIG. 7(a) graphically demonstrates the dissolution profile of zoledronic acid and C10 for tablets in EXP 1427 and 1428. FIG. 7(b) graphically demonstrates the dissolution profile of zoledronic acid and C10 for tablets in EXP 1427. FIG. 7(c) graphically demonstrates the dissolution profile of zoledronic acid and C10 for tablets in EXP 1428.

FIGS. 8(a) and 8(b) graphically demonstrate the dissolution profile of alendronate and C10 in tablets including sorbitol. FIG. 8(c) demonstrates the first derivative analysis of alendronate and C10 for tablets including sorbitol.

FIG. 9(a) graphically demonstrates the dissolution profile of acyline and C10 for tablets including sorbitol. FIG. 9(b) demonstrates the first derivative analysis of acyline and C10 for tablets including sorbitol.

FIG. 10 graphically shows the dissolution profile of the immediate co-release formulation of octreotide acetate and C10.

FIG. 11 graphically shows the dissolution profile of the non-co-release formulation of octreotide acetate and C10.

FIG. 12 graphically shows the dissolution profile of the extended co-release formulation of octreotide acetate and C10.

FIG. 13 graphically shows the comparison dissolution profile of the immediate co-release formulation, non-co-release formulation and extended co-release formulation of octreotide acetate and C10.

FIG. 14 graphically shows the comparison octreotide plasma concentration profile of the immediate co-release formulation, non-co-release formulation and extended co-release formulation of octreotide acetate and C10.

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “consists essentially of” (and grammatical variants), as applied to the compositions of this invention, means the composition can contain additional components as long as the additional components do not materially alter the composition. The term “materially altered,” as applied to a composition, refers to an increase or decrease in the therapeutic effectiveness of the composition of at least about 20% or more as compared to the effectiveness of a composition consisting of the recited components.

Unless otherwise defined, all terms, including technical and scientific terms used in the description, have the same meaning as commonly understood by one of ordinary skill in the art to which this invention belongs.

Unless the context indicates otherwise, it is specifically intended that the various features of the invention described herein can be used in any combination. For example, features described in relation to one embodiment may also be applicable to and combinable with other embodiments and aspects of the invention.

Moreover, the present invention also contemplates that in some embodiments of the invention, any feature or combination of features set forth herein can be excluded or omitted.

All patents, patent applications and publications referred to herein are incorporated by reference in their entirety. In case of a conflict in terminology, the present specification is controlling.

### I. Pharmaceutical Compositions

One aspect of the invention provides pharmaceutical compositions which are effective in providing therapeutically effective blood levels of a therapeutically active ingredient to a subject when administered to a gastrointestinal tract. The pharmaceutical compositions comprise, consist essentially of, or consist of: (i) a therapeutically effective amount of a therapeutically active ingredient; (ii) at least one water soluble enhancer; and (iii) a saccharide.

The investigators of the present invention discovered two important factors for maximizing the bioavailability of the active ingredient after oral administration of the pharmaceutical compositions described herein. The first is that the therapeutically active ingredient and the enhancer should be concurrently released at a substantially similar rate after the pharmaceutical composition enters the intestine of a subject. The second is that this release should occur rapidly. As a result of these two important factors, the interaction between the enhancer and the therapeutically active ingredient in the gastrointestinal tract may be maximized, which results in the most favorably improved bioavailability of the therapeutically active ingredient. The improved bioavailability allows the use of lower doses than previously needed and/or achievement of more effective treatment for the same dose. The investigators of the present application also observed that the release rate for the therapeutically active ingredient and the enhancer in vivo may be predicted by measuring the dissolution rate and/or disintegration rate for the therapeutically active ingredient and the enhancer from the dosage form in vitro.

As used herein, the term “rapid release rate” is defined as an in vitro dissolution of at least 80% of the therapeutically active ingredient and the enhancer from a dosage form without coating in 20 minutes. In other embodiments, the term “rapid release rate” is defined as an in vitro dissolution of at least 80% of the therapeutically active ingredient and the enhancer from a dosage form with a coating (e.g., an enteric coating or other type of delayed release or sustained release coating) in 20 minutes. In one embodiment, the dissolution is carried out in 900 mL pH 6.8 phosphate buffer at 37° C. with a USP Paddle Apparatus at 50 rpm. In one embodiment, the dissolution assay includes a preliminary step of acid treatment (e.g., 2 hrs in 0.1 N HCl). The term “dosage form without coating” refers to a dosage form comprising, consisting essentially of, or consisting of the pharmaceutical composition of the invention in the absence of any type of coating on the dosage form that would modulate the rate of release of the components of the dosage form (e.g., a delayed release or sustained release coating). In one embodiment, the dosage form is a tablet. Alternatively, the rapid release rate is defined as an in vitro dissolution of at least 95% of the therapeutically active ingredient and the enhancer from a dosage form without coating in 40 minutes. In another embodiment, the rapid release rate is defined as an in vitro dissolution of at least 70% of the therapeutically active ingredient and the enhancer from a dosage form with a coating in 40 minutes, e.g., at least about 75% or 80% in 40 minutes.

As used herein, the term “substantially similar release” is defined as a ratio of the time for a percentage of the therapeutically active ingredient to be released from a dosage form without coating to the time for the same percentage of the enhancer to be released in the range of about 1.3 to about 0.7. In other embodiments, the term “substantially similar release” is defined as a ratio of the time for a percentage of the therapeutically active ingredient to be released from a dosage form with a coating (e.g., an enteric coating or other type of delayed release or sustained release coating) to the time for the same percentage of the enhancer to be released in the range of about 1.3 to about 0.7. In one embodiment, the dissolution is carried out in 900 mL pH 6.8 phosphate buffer at 37° C. with a USP Paddle Apparatus at 50 rpm. In one embodiment, the dissolution assay includes a preliminary step of acid treatment (e.g., 2 hrs in 0.1 N HCl). For example, if zoledronic acid (therapeutically active ingredient) has a dissolution of 80% in about 20 minutes, sodium caprate (enhancer) must have a dissolution of 80% in the range of about 14 minutes to 26 minutes to be substantially similar. In one embodiment, the ratio is in the range of about 1.1 to about 0.9. For example, if zoledronic acid (therapeutically active ingredient) has a dissolution of 80% in about 20 minutes, sodium caprate (enhancer) must have a dissolution of 80% in the range of about 18 minutes to about 22 minutes.

In one embodiment, the therapeutically active ingredient and the enhancer in a dosage form without coating have a substantially similar dissolution of at least about 95% in less than about 40 minutes in pH 6.8 phosphate buffer at 37° C. In another embodiment, the therapeutically active ingredient and the enhancer in a dosage form without coating have a substantially similar dissolution of at least about 95% in less than about 30 minutes in pH 6.8 phosphate buffer at 37° C. Further, in one embodiment, the therapeutically active ingredient and the enhancer in a dosage form without coating have a substantially similar dissolution of at least about 80% in less than about 20 minutes in pH 6.8 phosphate buffer at 37° C. In another embodiment, the therapeutically active ingredient and the enhancer in a dosage form without coating have a substantially similar dissolution of at least about 80% in less than about 18 minutes in pH 6.8 phosphate buffer at 37° C. In further embodiments, this dissolution rates are met with a coated dosage form.

Alternatively, the dissolution profile of the therapeutically active ingredient and the enhancer may also be compared using f1 and f2 values. Moore and Flanner (Pharm. Tech. 20(6): 64-74, 1996) proposed a model independent mathematical approach to compare the dissolution profile of two components using two factors, f1 and 12, as shown in the following formula.

f1={[Sₜ₌₁ⁿ(Rₜ−Tₜ)]/[Sₜ₌₁ⁿRₜ]}·100

f2=50·log {[1+(1/n)Sₜ₌₁ⁿ(Rₜ−Tₜ)²]^{-0.5}·100}

Here Rₜ and Tₜ are the cumulative percentage dissolved at each of the selected n time points of the reference and test product respectively. Relative standard deviation (RSD or RSD) is the absolute value of the coefficient of variation, often expressed as a percentage. The formula for calculating the % RSD may be described as: % Relative standard deviation=((standard deviation of array X)/(mean of array X))×100; X is the number of samples taken for each time points. The factor f1 is proportional to the average difference between the two profiles, where as factor f2 is inversely proportional to the average squared difference between the two profiles, with emphasis on the larger difference among all the time-points. The factor f2 measures the similarity between the two profiles. Because of the nature of the measurement, f1 is described as a difference factor, and f2 as a similarity factor.

When the two dissolution profiles are identical, f1=0 and f2=100. An average difference of 10% at all measured time points results in a f2 value of 50. The FDA has set a public standard of f2 value between 50-100 to indicate similarity between dissolution profiles of two tablets. It is generally accepted that an f1 value of less than 15 indicates similarity.

The data contained herein allows one to define a set of data inclusion criteria that are appropriate to determine whether a dosage form releases the therapeutically active ingredient rapidly enough and in sufficient conjunction with the enhancer to allow appropriate maximization of the effect of the enhancer. The following criteria apply: (1) at least 6 tablets should be used for each profile determination; (2) the mean dissolution values can be used to estimate the similarity factors (to use mean data, the % coefficient of variation at the earliest point should not be more than 30% and at other time points should not be more than 20%; and (3) at least 4 dissolution values must be used in the calculation, none of which can be 0, and only one of which can be greater than 85% dissolution.

The same time points must be used for both the therapeutically active ingredient and the enhancer. Therefore it may not be possible to satisfy all the criteria for dissolution of both the enhancer and therapeutically active ingredient simultaneously. In one example, for a formulation where non-co-release occurs it may be necessary for one of the profiles (for the faster component) to have more then 1 value above 85%. In another example, for a formulation where non-co-release occurs it may not be possible for both profiles to satisfy the % RSD requirements at the same time points, due to significantly lower percent dissolved of one component over the other at that timepoint.

The Moore and Flanner model independent mathematical approach has been adapted to compare the dissolution profile of enhancer and therapeutically active ingredient and define co-release. Substantially similar co-release is defined herein as a f1 value of less than 15. For quality control purposes for comparisons of tablets containing the same active ingredient with different formulations, a f1 value of less than 15 is generally accepted to indicate similarity.

A f2 value of 50-100 is defined herein to indicate substantially similar co-release of the therapeutically active ingredient and enhancer. The inventors are not aware of anyone using this sort of approach to optimize and ensure that an oral absorption enhancer is appropriately formulated with an active drug substance to assure appropriate enhancer performance.

In some embodiments, for f1 and f2 analysis, the number of time points may be 4, 5, 6, 7, 8, or 9 or more. It is understood by one skilled in the art that, even with the criteria defined above, f1 and f2 values may be manipulated by changing the number and/or time intervals of sample points, their location on the dissolution curve, and other variants. Thus, the f1 and f2 calculations are tools to compare the dissolution profile of different formulations and demonstrate the properties of the pharmaceutical compositions described herein. In addition, the f1 and f2 calculations may also be used as tools to compare enhancer and therapeutically active ingredient release within one formulation. The scope of the invention should not be limited to the exact value of f1 and f2.

In one embodiment of the invention, the f1 value for the dissolution profile of the enhancer and the therapeutically active ingredient is less than about 25, e.g., less than about 20, 15, 10 or 5. In other embodiments of the invention, the f2 value for the dissolution profile of the enhancer and the therapeutically active ingredient is at least about 50, e.g., at least about 55, 60, 65, 70, 75, 80, 85, 90 or 95.

For instantly soluble pharmaceutical compositions, the disintegration rate may predict the dissolution behavior because the disintegration of the dosage form of the pharmaceutical composition may be the rate-limiting step to dissolution. The disintegration test used to test the dosage form of the pharmaceutical compositions described herein is carried out as described in the EP 2.9.1 monograph Disintegration of Tablets and Capsules for uncoated tablets. The compendia recommendation is to use water. The temperature for the test is 37 degrees Celsius. According to some aspects of the present invention, the pharmaceutical compositions described herein provide a relatively fast disintegration rate. In one embodiment, the pharmaceutical composition in a dosage form without coating has a disintegration time of less than about 15 minutes at 37° C. In another embodiment, the pharmaceutical composition in a dosage form without coating has a disintegration time of less than about 10 minutes at 37° C.

As used herein, the term “therapeutically active ingredient,” which is interchangeably used with “active ingredient”, refers to any chemical compound, complex or composition that has a beneficial biological effect, preferably a therapeutic effect in the treatment of a disease or abnormal physiological condition. The terms also encompass pharmaceutically acceptable, pharmacologically active derivatives of those active agents specifically mentioned herein, including, but not limited to, salts, esters, amides, prodrugs, active metabolites, isomers, fragments, analogs, and the like. When the terms “therapeutically active ingredient” or “active ingredient” is used and when a particular active agent is specifically identified, it is to be understood that applicants intend to include the active agent per se as well as pharmaceutically acceptable, pharmacologically active salts, esters, amides, prodrugs, active metabolites, isomers, fragments, analogs, etc.

The therapeutically active ingredient of the present invention includes any active ingredient that is appropriate for administration via the oral route to an animal including a human. The term “active ingredient” also explicitly includes those entities that are poorly absorbed via the oral route including hydrophilic drugs or macromolecular drugs such as peptides, proteins, oligosaccharides, polysaccharides or hormones including, but not limited to, insulin, calcitonin, calcitonin gene regulating protein, atrial natriuretic protein, colony stimulating factor, betaseron, erythropoietin (EPO), interferons, somatropin, somatotropin, somatostatin, insulin-like growth factor (somatomedins), luteinizing hormone releasing hormone (LHRH), tissue plasminogen activator (TPA), thyrotropin releasing hormone (TRH), growth hormone releasing hormone (GHRH), antidiuretic hormone (ADH) or vasopressin and analogues thereof such as for example desmopressin, parathyroid hormone (PTH), oxytocin, estradiol, growth hormones including human growth hormone, leuprolide acetate, goserelin acetate, naferelin, buserelin, factor VIII, interleukins such as interleukin-2, and analogues thereof and anti-coagulant agents such as heparin, heparinoids, low molecular weight heparin (LMWH), hirudin, and analogues thereof, bisphosphonates including alendronate, clodronate, etidronate, incadronate, ibandronate, minodronate, pamidronate, risedronate, tiludronate and zoledronate, pentasaccharides including anti-coagulant pentasaccharides, antigens, adjuvants and the like. In some embodiments, the active ingredient is Glucagon-like peptide 1 (GLP-1), analogues or agonists thereof, such as for example exenatide, liraglutide. In some embodiments, the therapeutically active ingredient is low molecular weigh heparin. In one embodiment, the low molecular weigh heparin is selected from parnaparin, fondaparinux, nardroparin, certroparin, tinzaparin, daltaparin, or enoxoparin.

In another embodiment, the therapeutically active ingredient is a hydrophilic drug. As used herein, the term “hydrophilic drug” is defined as drug with solubility in water greater than 1 percent (w/v) and that is practically insoluble in nonpolar organic solvents such as ethyl acetate, methylene chloride, chloroform, toluene, or hydrocarbons.

In one embodiment, the active ingredient is a bisphosphonate or a pharmaceutically acceptable salt thereof. In other embodiments, the active ingredient is selected from alendronate, clodronate, etidronate, incadronate, ibandronate, minodronate, pamidronate, risedronate, tiludronate, zoledronate, or a pharmaceutically acceptable salt thereof. In some embodiments, the active ingredient is alendronic acid or a pharmaceutically acceptable salt thereof. In some embodiments, the active ingredient is zoledronic acid, or a pharmaceutically acceptable salt thereof.

In one embodiment, the therapeutically active agent may include GnRH related compounds, including both GnRH antagonists and GnRH agonists. In some embodiments, the present invention may be applied to GnRH antagonists. In some embodiments, the present invention includes, but is not limited to, the following GnRH antagonists, acyline (Ac-D2Nal-D4 Cpa-D3 Pal-Ser4Aph(Ac)-D4Aph(Ac)-Leu-ILys-Pro-DAla-NH₂), Acetyl-β-[2-Naphthyl]-D-Ala-D-p-Chloro-Phe-β-[3-Pyridyl]-D-Ala-Ser-Nε-[Nicotinoyl]-Lys-Nε-[Nicotinoyl]-D-Lys-Leu-Nε-[Isopropyl]-Lys-Pro-D-Ala-NH₂ (also referred to herein as Antide), acetyl-D2Nal1, D4C1Phe2, D3 Pal3, ARg5, Dglu6 (AA) (also referred to herein as NalGlu), acetyl-D2Nal-D4CIPhe-D3 Pal-Ser-Aph(Ac)-D-Aph(Ac)-Leu-Lys(lpr)-Pro-D-Ala-NH₂, Abarelix (Specialty European Pharma, Dusseldorf, Germany), NaI-Lys, Synarel, (Searle, Peapack, N.J.), Ganirelix (Orgalutron/Antagon) (Organan, West Orange, N.J.), Cetrorelix I (Aeterna Zentaris Inc, Frankfurt, Germany), Cetrotide, Azaline B, new generation long-acting GnRH analogues incorporating p-ureido-phenylalanines at positions 5 and 6 (such as Degarelix), FE200486, Ac-D2Nal-D4 Cpa-D3 Pal-Ser-4Aph(L-hydroorotyl)-D4Aph(carbarnoyl)-Leu-ILys-Pro-DAla-NH₂ (the acetate salt of which is FE200486), Ac-D2NaI-D4 Cpa-D3 Pal-Ser-4Aph(Atz)-D4Aph(Atz)-Leu-ILys-Pro-DAla-NH₂ wherein Atz is 3′-amino-1H-1′,2′,4′-triazol-5′-yl, and the antagonists described in U.S. Pat. Nos. 5,506,207, 5,821,230, 5,998,432, 6,156,772, 6,156,767, 6,150,522, 6,150,352, 6,147,088, 6,077,858, 6,077,847, 6,025,366, 6,017,944, 6,004,984, 6,214,798, and 6,875,843. In some embodiments, at least one GnRH antagonist is selected from the group consisting of acyline, abarelix, azaline B, cetrorelix, ganirelix, teverelix, degarelix, antide, orntide and GnRH antagonists described in U.S. Pat. No. 7,098,305.

In some embodiments, the active ingredient is a HDAC inhibitor. As used herein, the terms “histone deacetylase” and “HDAC” are intended to refer to any one of a family of enzymes that remove acetyl groups from the α, ε-amino groups of lysine residues at the N-terminus of a histone. Unless otherwise indicated by context, the term “histone” is meant to refer to any histone protein, including H1, H2A, H₂B, H3, H4, and H5, from any species. Histone deacetylases may include class I and class II enzymes, and may also be of human origin, including, but not limited to, HDAC-1, HDAC-2, HDAC-3, HDAC-4, HDAC-5, HDAC-6, HDAC-7, and HDAC-8. In some embodiments, the histone deacetylase is derived from a protozoal, bacterial or fungal source.

As used herein, the terms “histone deacetylase inhibitor” and “HDAC inhibitor” are intended to refer to a compound which is capable of interacting with a histone deacetylase and inhibiting its enzymatic activity. The phrase “inhibiting histone deacetylase enzymatic activity” means reducing the ability of a histone deacetylase to remove an acetyl group from a histone. In some embodiments, such reduction of histone deacetylase activity is at least about 50%, at least about 75%, or at least about 90%. In other embodiments, histone deacetylase activity is reduced by at least 95% or at least 99%. Suitable HDAC inhibitors include, but not limited to, short-chain fatty acids such as butyrate, phenylbutyrate, pivaloyloxymethyl butyrate, N-Hydroxy-4-(3-methyl-2-phenyl-butyrylamino)-benzamide,4-(2,2-Dimethyl-4-phenylbutyrylamino)-N-hydroxybenzamide, valproate and valproic acid; hydroxamic acids and their derivatives such as suberoylanilide hydroxamic acid (SAHA) and its derivatives, oxamflatin, M-carboxycinnamic acid bishydroxamide, suberic bishydroxamate (SBHA), nicotinamide, scriptaid (SB-556629), scriptide, splitomicin, lunacin, ITF2357, A-161906, NVP-LAQ824, LBH589, pyroxamide, CBHA, 3-Cl-UCHA, SB-623, SB-624, SB-639, SK-7041, propenamides such as MC 1293, aroyl pyrrolyl hydroxyamides such as APHA Compound 8, and trichostatins such as trichostatin A and trichostatin C; cyclic tetrapeptides such as trapoxins, romidepsin, HC-toxin, chlamydocin, diheteropeptin, WF-3161, Cyl-1, Cyl-2, apicidin, depsipeptide (FK228), FR225497, FR901375, spiruchostatins such as spiruchostatin A, spiruchostatin B and spiruchostatin C, salinamides such as salinamide A and salinamide B, and cyclic-hydroxamic-acid-containing peptides (CHAPs); benzamides such as M344, MS-275, CI-994 (N-acetyldinaline), tacedinaline and sirtinol; tricyclic lactam and sultam derivatives; organosulfur compounds such as diallyl disulfide and sulforaphane; electrophilic ketones such as α-ketoamide and trifluoromethylketone; pimeloylanilide o-aminoanilide (PAOA); depudecin; psammaplins; tubacin; curcumin; histacin; 6-Chloro-2,3,4,9-tetrahydro-1H-carbazole-1-carboxamide, CRA-024781; CRA-026440; CG1521; PXD101; G2M-777, CAY10398, CTPB and MGCDO103. The term “HDAC inhibitor” also includes all analogs and forms thereof including optically pure enantiomers or mixtures, racemic or otherwise, of enantiomers as well as all pharmaceutically acceptable derivative forms thereof. In one embodiment, the HDAC inhibitor is depsipeptide.

In one embodiment, the active ingredient is selected from somatostatin, sandostatin LAR (octreotide acetate), Forteo (teriparatide), Gemzar (gemcitabine), ubicin (daptomycin), Treanda (bendamustine), vitamin B12 (cyanocobalamin), Vitamin D3, Avonex (Interferon β-1a), velcade (bortezomib), and human growth hormone.

In one embodiment, the active ingredient is an iron complex. As used herein, the “iron” complex include iron (Fe) in any of its oxidative states and in combination with any salt. “Ferrous” refers to iron with a +2 charge (also denoted in the art as Fe²⁺, Fe⁺⁺, iron (II)). “Ferric” refers to iron with a +3 charge (also denoted in the art as Fe³⁺, Fe⁺⁺⁺, iron (III)). Exemplary ferrous salts and ferric salts include, but are not limited to ferrous and ferric sulfate, fumarate, succinate, gluconate, etc. Other exemplary complexes also include those described in PCT Publications No. WO/2005/041928. In one embodiment, the “iron” complex may be in a form of chelates or salts. Examples include, but are not limited to, ferric pyrophosphate and sodium iron EDTA.

As used herein, the term “active ingredient” includes all forms thereof including optically pure enantiomers or mixtures, racemic or otherwise, of enantiomers as well as derivative forms such as, for example, salts, acids, esters and the like. The active ingredient may be provided in any suitable phase state including as a solid, liquid, solution, suspension and the like. When provided in solid particulate form, the particles may be of any suitable size or morphology and may assume one or more crystalline, semi-crystalline and/or amorphous forms.

As used herein, a “therapeutically effective amount of a therapeutically active ingredient” refers to an amount of active ingredient that elicits a therapeutically useful response in an animal. In some embodiments, the animal is a mammal. In some embodiments, the animal is a human.

As used herein, the term “enhancer” refers to a water soluble compound (or a mixture of compounds) which is capable of enhancing the transport of a therapeutically active ingredient (e.g., absorption), particularly a hydrophilic and/or macromolecular therapeutically active ingredient across the gastrointestinal tract in an animal such as a human. The term “water soluble” as used herein is defined as a compound that is soluble or miscible in water at a concentration of about 0.5 mg/ml, e.g., 1 mg/ml or 10 mg/ml at room temperature. Enhancers include, without limitation, surfactants, fatty acids, medium chain glycerides, steroidal detergents, acyl carnitines and alkanoylcholines, N-acetylated α-amino acids and N-acetylated non-α-amino acids such as sodium 8-[N-(2-hydroxybenzoyl)amino]caprylate (SNAC) and sodium 10-[N-(2 hydroxybenzoyl)amino]decanoate (SNAD), and chitosans and other mucoadhesive polymers as well as salts and derivatives of these compounds. In some embodiments, an enhancer is a water soluble compound that increases the bioavailability of a therapeutically active ingredient by at least 5%, e.g., at least 10, 20, 30, 40, or 50%, when orally administered in a pharmaceutical composition comprising the therapeutically active ingredient as compared to a pharmaceutical composition that does not comprise the enhancer.

In some embodiments, the enhancer is a medium chain fatty acid or a salt, ester, ether, or derivative of a medium chain fatty acid and which has a carbon chain length of from 4 to 20 carbon atoms. In some embodiments, the enhancer is medium chain fatty acid or a salt, ester, ether, or derivative of a medium chain fatty acid and which has a carbon chain length of from 6 to 20 carbon atoms. In some embodiments, the carbon chain length is from 8 to 14 carbon atoms. In some embodiments, the enhancer is a medium chain fatty acid or a salt, ester, ether, or derivative of a medium chain fatty acid and which has a carbon chain length of from 6 to 20 carbon atoms; with the provisos that (i) where the enhancer is an ester of a medium chain fatty acid, said chain length of from 6 to 20 carbon atoms relates to the chain length of the carboxylate moiety, and (ii) where the enhancer is an ether of a medium chain fatty acid, at least one alkoxy group has a carbon chain length of from 6 to 20 carbon atoms. In another embodiment, the enhancer is a medium chain fatty acid or a salt, ester, ether, or derivative of a medium chain fatty acid which is solid at room temperature and which has a carbon chain length of from 8 to 14 carbon atoms; with the provisos that (i) where the enhancer is an ester of a medium chain fatty acid, said chain length of from 8 to 14 carbon atoms relates to the chain length of the carboxylate moiety, and (ii) where the enhancer is an ether of a medium chain fatty acid, at least one alkoxy group has a carbon chain length of from 8 to 14 carbon atoms.

In some embodiments, the enhancer is a sodium salt of a medium chain fatty acid. In another embodiment, the medium chain fatty acid has a carbon chain length of from 8 to 14 carbon atoms. In some embodiments, the sodium salt is solid at room temperature. In another embodiment, the enhancer is selected from the group consisting of sodium caprylate, sodium caprate (also described as “C10”) and sodium laurate. In some embodiments, the enhancer is sodium caprate. The enhancer is further described in U.S. Patent Application Publication No. 2003/0091623, which is incorporated by reference in its entirety. In some embodiments, the enhancer is the only absorption enhancer present in the composition.

As used herein, a “derivative of a medium chain fatty acid” comprises a fatty acid derivative having at least one carbon chain of from 4 to 20 carbon atoms in length. This carbon chain may be characterized by various degrees of saturation. In other words, the carbon chain may be, for example, fully saturated or partially unsaturated (i.e., containing one or more carbon-carbon multiple bonds). The term “fatty acid derivative” is meant to encompass acyl derivatives such as esters, acid halides, anhydrides, amides and nitrites, and also ethers and glycerides such as mono-, di- or tri-glycerides. The term “fatty acid derivative” is meant to further encompass medium chain fatty acids wherein the end of the carbon chain opposite the acid group (or derivative) is also functionalized with one of the above mentioned moieties (i.e. ester, acid halide, anhydride, amide, nitrile, ether and glyceride moieties). Such difunctional fatty acid derivatives thus include for example diacids and diesters (the functional moieties being of the same kind) and also difunctional compounds comprising different functional moieties, such as amino acids and amino acid derivatives (for example a medium chain fatty acid, or an ester or a salt thereof, comprising an amide moiety at the opposite end of the fatty acid carbon chain to the acid (or ester or salt thereof). In some embodiments, the derivative of a medium chain fatty acid has at least 20% of the absorption enhancing activity of the medium chain fatty acid from which it is derived, e.g., at least 30%, 40%, 50%, 60%, 70%, 80%, or more of the absorption enhancing activity.

Any suitable amount of enhancer may be incorporated in the compositions described herein. However, in some embodiments, the weight percentage of the enhancer is at least about 50 percent of the total weight of the pharmaceutical composition in one dosage unit. In another embodiment, the weight percentage of enhancer is at least about 60 percent of the total weight of the pharmaceutical composition in one dosage unit. In one embodiment, the amount of enhancer is at least about 2.0 mmol in one dosage unit. In some embodiments, the amount of enhancer is at least about 2.5 mmol in one dosage unit. Further, in one embodiment, the amount of enhancer is at least about 3.5 mmol in one dosage unit. In some embodiments, the amount of enhancer (e.g., sodium caprate) is at least about 400 mg (about 2.06 mmol of sodium caprate). In one embodiment, the amount of enhancer (e.g., sodium caprate) is at least about 550 mg (about 2.8 mmol of sodium caprate). In some embodiments, the amount of enhancer (e.g., sodium caprate) is at least about 700 mg (about 3.6 mmol of sodium caprate).

As used herein, a “therapeutically effective amount of an enhancer” refers to an amount of enhancer that allows for uptake of therapeutically effective amounts of the therapeutically active ingredient via oral administration. It has been shown that the effectiveness of an enhancer in improving the gastrointestinal absorption of poorly absorbed drugs is dependent on the site of administration, the site of optimum delivery being dependent on the drug and enhancer.

Saccharides are widely used in pharmaceutical formulations as a diluent but are not known to have disintegration properties. However, it has been found that formulations including saccharides (e.g., sorbitol or mannitol) disintegrate significantly faster than formulations without saccharides. When incorporated with an effective amount of a water soluble bioavailability enhancer, tablets made with a saccharide generally disintegrate more quickly. It has even been found surprisingly that some enhancer formulations made with binders with disintegration properties disintegrate slower than enhancer formulations with saccharides. The presence of saccharides in pharmaceutical compositions of the present invention may also affect the dissolution rate of the active ingredient and water soluble enhancer components. It has been found that the presence of a saccharide (e.g., sorbitol) can provide a substantially similar dissolution rate for the active agent and water soluble enhancer, where the dissolution rates of these components in the absence of saccharide may differ distinctly. In particular, the presence of a saccharide (e.g., sorbitol) in a formulation with a bisphosphonate (e.g., alendronate or zoledronic acid) and a water soluble enhancer (e.g., a fatty acid enhancer as defined herein, such as a C₁₀ fatty acid, e.g., sodium caprate) can facilitate substantially similar dissolution rates of the bisphosphonate active ingredient and the water soluble enhancer.

Any suitable saccharide may be included in the composition of the present invention. As used herein, the “saccharides” used in the invention include sugar alcohols, monosaccharides, di-saccharides and oligosaccharides. Exemplary sugar alcohols include, but are not limited to, xylitol, mannitol, sorbitol, erythritol, lactitol, pentitol and hexitol. Exemplary monosaccharides include, but are not limited to, glucose, fructose, aldose and ketose. Exemplary di-saccharides include, but are not limited to, sucrose, isomalt, lactose, trehalose, and maltose. Exemplary oligosaccharides include, but are not limited to, maltotriose, raffinose and maltotetraose. In some embodiments, the saccharide is sorbitol, mannitol, or xylitol. In some embodiments, the saccharide is sorbitol. In some embodiments, the saccharide is sucrose. In some preferred embodiments, saccharides are incorporated with water soluble enhancers such as fatty acid enhancers, such as C₄-C₂₀, e.g., C₈-C₁₄, e.g., C₁₀ fatty acid enhancers or salts or derivatives thereof such as sodium caprate. The inclusion of saccharides is also preferred for compositions comprising bisphosphonates such as alendronate or zoledronic acid. In some embodiments, compositions comprising saccharides (e.g., saccharides as described above, such as sorbitol or mannitol) in combination with fatty acid enhancers (e.g., as described above) and a bisphosphonate active ingredient (e.g., as described above) are particularly preferred. In particular it has been found that the dissolution rate of zoledronic acid and C₁₀ fatty acids is significantly improved in the presence of sorbitol.

Any suitable amounts of saccharide may be added in the compositions of the present invention. In some embodiments of the present invention, the ratio of the enhancer and saccharide may be adjusted to achieve a desired dissolution rate and/or compressibility of the resulting pharmaceutical composition. In some embodiments, the ratio of weight percentage of the enhancer and saccharide is about 2:1 to 20:1, e.g., about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1 or any range therein. However, according to some embodiments, the ratio of the weight percentage of the enhancer and saccharide is about 3:1 to 6:1. Yet, in another embodiment, the ratio of the weight percentage of the enhancer and saccharide is about 5:1. In one embodiment, the ratio of the weight percentage of the enhancer and saccharide is about 4:1.

Any suitable grade of saccharide may be used in the composition of the present invention. However, in some embodiments, the selection of the grade of saccharide may be dependent upon the particle size distribution (PSD) of a specific grade of saccharide. Further, in another embodiment, the specific grade of the saccharide may affect the characteristics of the resulting pharmaceutical composition such as dissolution rate or compressibility. In some embodiments, the selection of the grade of saccharide is dependent upon the PSD of other excipients and the therapeutically active ingredient. In some embodiments, the saccharide is Parteck 150 directly compressible sorbitol. In other embodiments, the saccharide is Parteck SI 400 (Merck KGaA, Darmstadt, Germany).

The pharmaceutical compositions of the invention can comprise one or more auxiliary excipients, such as for example rate-controlling polymeric materials, diluents, lubricants, disintegrants, plasticizers, anti-tack agents, opacifying agents, glidants, pigments, flavorings and such like. As will be appreciated by those skilled in the art, the exact choice of excipients and their relative amounts will depend to some extent on the final dosage form.

Suitable diluents include, for example, pharmaceutically acceptable inert fillers such as microcrystalline cellulose, lactose, dibasic calcium phosphate, saccharides, and/or mixtures of any of the foregoing. Examples of diluents include microcrystalline cellulose such as that sold under the Trademark Avicel (FMC Corp., Philadelphia, Pa.), for example, Avicel™ pH101, Avicel™ pH102 and Avicel™ pH112; lactose such as lactose monohydrate, lactose anhydrous and Pharmatose DCL21; dibasic calcium phosphate such as Emcompress; mannitol; starch; sorbitol; sucrose; glucose; and combinations and mixtures thereof.

Suitable lubricants, including agents that act on the flowability of the powder to be compressed are, for example, colloidal silicon dioxide such as Aerosil™ 200; talc; stearic acid; magnesium stearate; calcium stearate; and combinations and mixtures thereof.

Suitable disintegrants include, for example, lightly crosslinked polyvinyl pyrrolidone, corn starch, potato starch, maize starch and modified starches, croscarmellose sodium, crospovidone, sodium starch glycolate, and combinations and mixtures thereof.

The term “rate controlling polymer material” as used herein includes hydrophilic polymers, hydrophobic polymers and mixtures of hydrophilic and/or hydrophobic polymers that are capable of controlling or retarding the release of the active ingredient from a solid oral dosage form of the present invention. Suitable rate controlling polymer materials include those selected from the group consisting of hydroxyalkyl cellulose such as hydroxypropyl cellulose and hydroxypropyl methyl cellulose; poly(ethylene) oxide; alkyl cellulose such as ethyl cellulose and methyl cellulose; carboxymethyl cellulose; hydrophilic cellulose derivatives; polyethylene glycol; polyvinylpyrrolidone; cellulose acetate; cellulose acetate butyrate; cellulose acetate phthalate; cellulose acetate trimellitate; polyvinyl acetate phthalate; hydroxypropylmethyl cellulose phthalate; hydroxypropylmethyl cellulose acetate succinate; polyvinyl acetaldiethylamino acetate; poly(alkylmethacrylate) and poly(vinyl acetate). Other suitable hydrophobic polymers include polymers and/or copolymers derived from acrylic or methacrylic acid and their respective esters, zein, waxes, shellac and hydrogenated vegetable oils. Particularly useful in the practice of the present invention are poly acrylic acid, poly acrylate, poly methacrylic acid and poly methacrylate polymers such as those sold under the Eudragit tradename (Rohm GmbH, Darmstadt, Germany) specifically Eudragit® L, Eudragit® S, Eudragit® RL, and Eudragit® RS coating materials and mixtures thereof. Some of these polymers can be used as delayed release polymers to control the site where the drug is released. They include poly methacrylate polymers such as those sold under the Eudragit tradename (Rohm GmbH, Darmstadt, Germany) specifically Eudragit® L, Eudragit® S, Eudragit® RL, and Eudragit® RS coating materials and mixtures thereof.

The pharmaceutical composition according to the present invention may be in a dosage form of a tablet, particulate, multi-particulate, capsule, pellet, mini-tablets, encapsulated pellet, encapsulated mini-tablets, encapsulated micro-particulate, or mucoadhesive forms (e.g., tablets or capsules). In one embodiment, the pharmaceutical composition may be in a dosage form (e.g., tablet) without a coating. In some embodiments, the pharmaceutical composition is in a delayed release dosage form which minimizes the release of the active ingredient and the enhancer in the stomach, and hence the dilution of the local enhancer concentration therein, and releases the drug and enhancer in the intestine. In other embodiments, the pharmaceutical composition is in a delayed release rapid onset dosage form. Such a dosage form minimizes the release of the active ingredient and enhancer in the stomach, and hence the dilution of the local enhancer concentration therein, but releases the active ingredient and enhancer rapidly once the appropriate site in the intestine has been reached, maximizing the delivery of the poorly permeable active ingredient by maximizing the local concentration of the active ingredient and enhancer at the site of absorption. In some dosage forms, the pharmaceutical composition is in the form of a tablet.

The term “tablet” as used herein includes, but is not limited to, immediate release (IR) tablets, sustained release (SR) tablets, matrix tablets, multilayer tablets, multilayer matrix tablets, extended release tablets, delayed release tablets and pulsed release tablets, any or all of which may optionally be coated with one or more coating materials, including polymer coating materials, such as enteric coatings, rate-controlling coatings, semi-permeable coatings and the like. The term “tablet” also includes osmotic delivery systems in which a drug compound is combined with an osmagent (and optionally other excipients) and coated with a semi-permeable membrane, the semi-permeable membrane defining an orifice through which the drug compound may be released. In some embodiments, the pharmaceutical composition of the present invention is selected from the group consisting of IR tablets, SR tablets, coated IR tablets, matrix tablets, coated matrix tablets, multilayer tablets, coated multilayer tablets, multilayer matrix tablets and coated multilayer matrix tablets. Yet, in some embodiments, the pharmaceutical composition is in an enteric coated tablet dosage form. In other embodiments, the pharmaceutical composition is in an enteric coated rapid onset tablet dosage form.

In some embodiments, the pharmaceutical composition of the present invention may be in a form of a capsule solid oral dosage form. In some embodiments, the capsule solid oral dosage form of the present invention is selected from the group consisting of instant release capsules, sustained release capsules, coated instant release capsules and coated sustained release capsules including delayed release capsules. Yet, in another embodiment, the capsule dosage form is an enteric coated capsule dosage form. In some embodiments, the capsule dosage form is an enteric coated rapid onset capsule dosage form.

The term “multiparticulate” as used herein means a plurality of discrete particles, pellets, mini-tablets and mixtures or combinations thereof. If the pharmaceutical composition is in a multiparticulate capsule, such hard or soft gelatin capsules can suitably be used to contain the multiparticulate. Alternatively, a sachet can suitably be used to contain the multiparticulate. If desired, the multiparticulate may be coated with a layer containing rate controlling polymer material. A multiparticulate oral dosage form according to some embodiments of the invention may comprise a blend of two or more populations of particles, pellets, or mini-tablets having different in vitro and/or in vivo release characteristics. For example, a multiparticulate oral dosage form may comprise a blend of an instant release component and a delayed release component contained in a suitable capsule.

Alternatively, the multiparticulate and one or more auxiliary excipients can be compressed into tablet form such as a multilayer tablet. In some embodiments, a multilayer tablet may comprise two layers containing the same or different levels of the same active ingredient having the same or different release characteristics. In another embodiment, a multilayer tablet may contain a different active ingredient in each layer. Such a tablet, either single layered or multilayered, can optionally be coated with a controlled release polymer so as to provide additional controlled release properties. Yet, in some embodiments, a multiparticulate dosage form of the present invention comprises a capsule containing delayed release rapid onset minitablets. In another embodiment, the multiparticulate dosage form comprises a delayed release capsule comprising instant release minitablets. In some embodiments, the multiparticulate dosage form comprises a capsule comprising delayed release granules. In another embodiment, the multiparticulate dosage form comprises a delayed release capsule comprising instant release granules.

In the case of any of the above-mentioned embodiments, a controlled release coating (e.g., an enteric coating) may be applied to the final dosage form (capsule, tablet, multilayer tablet, etc.). The controlled release coating may typically comprise a rate controlling polymer material as defined above. The dissolution characteristics of such a coating material may be pH dependent or independent of pH.

In some embodiments, the pharmaceutical composition can be coated or uncoated. In some embodiments, the pharmaceutical composition is uncoated.

### II. Methods of Providing the Pharmaceutical Compositions in a Single Dosage Form

Another aspect of the present invention provides methods of providing a pharmaceutical composition described herein in a single dosage unit with a patient acceptable size. The methods comprise directly compressing or dry granulating the enhancer without adding any moisture agent before preparing the dosage form. In one embodiment, the methods described herein further comprise mixing the compressed or granulated enhancer with the therapeutically active ingredient and the saccharide. In another embodiment, the enhancer is compressed or granulated by itself. In one embodiment, the patient acceptable size is no more than about 1.2 g/per dosage. In some embodiments, the patient acceptable size is no more than about 1.0 g/per dosage.

As used herein, the process of “directly compressing” refers to a process where the powdered components included in the solid dosage form are compressed directly without modifying their physical nature. In some embodiments, the direct compression process does not include any moisture agent.

As used herein, the process of “dry granulating” is a process of mixing the ingredients, slugging the ingredients, dry screening, lubricating and finally compressing the ingredients. In some embodiments, the mixing step may optionally include a lubricant. According to some embodiments of the present invention, the dry granulation process does not include any moisture agent. The dry granulation process usually applies when a component, either the active ingredient or the excipients, has sufficient cohesive properties to be tableted. It is preferred that dry granulation is used in the preparation of pharmaceutical compositions according to the present invention. In particular, the use of dry granulation is preferred when the composition comprises water soluble enhancers, such as fatty acid enhancers, such as C₄-C₂₀, e.g., C₈-C₁₄, e.g., C₁₀ fatty acid enhancers or salts or derivatives thereof such as sodium caprate. The use of dry granulation is also preferred for compositions comprising bisphosphonates such as alendronate or zoledronic acid. The use of dry granulation processes can provide improved bioavailability and faster release of the active agent from pharmaceutical compositions, especially in these preferred situations. This improved bioavailability may be due to the ability to incorporate more sodium caprate in one tablet prepared using dry granulation and the more rapid dissolution afforded by the tablets prepared by dry granulation. Therefore, dry granulation is the preferred manufacturing technique for enhancing absorption via administration of water soluble enhancers.

### III. Methods of Treatment

A further aspect of the present invention provides methods for the treatment and/or prevention of a medical condition which is effective in providing therapeutically effective blood levels of a therapeutically active ingredient to a subject when administered to a gastrointestinal tract of the subject, comprising administering orally to the subject a pharmaceutical composition described herein. Pharmaceutical compositions for use in the treatment and/or prevention of a medical condition are also envisaged, particularly where the use comprises administration of the composition to the gastrointestinal tract of a subject to provide therapeutically effective blood levels of a therapeutically active ingredient.

In some embodiments, the therapeutically active ingredient is a bisphosphonate compound. The medical condition can be any condition for which a bisphosphonate compound may provide a therapeutic, prophylactic, or diagnostic benefit. Exemplary medical conditions include, but are not limited to osteoporosis, rheumatoid arthritis, bone fracture, excessive bone resorption, bone cancer, and a combination thereof.

In one embodiment, the therapeutically active ingredient is a GnRH antagonist. The medical condition can be any condition for which a GnRH antagonist may provide a therapeutic, prophylactic, or diagnostic benefit. Exemplary medical conditions include, but are not limited to, sex hormone dependent diseases such as benign prostate hyperplasia, prostate cancer, estrogen-dependent breast cancer, endometrial cancer, ovarian cancer, endometriosis and precocious puberty, and contraception in a human or animal subject.

In one aspect, the therapeutically active ingredient is a peptide or protein active ingredient. The medical condition may be any condition for which a peptide or protein provides a therapeutic, prophylactic, or diagnostic benefit. Examples of medical conditions that can be treated, prevented, or diagnosed by the present invention include, without limitation, congestive heart failure, sepsis, vaccines (e.g., Lyme disease vaccine), chronic hepatitis C, cancer (e.g., hairy cell leukemia, chronic myelogenous leukemia, malignant melanoma, cutaneous T-cell lymphoma, HER2-positive metastatic breast cancer, acute lymphoblastic leukemia, B-cell chronic lymphocytic leukemia), AIDS-related Kaposi's sarcoma, venereal or genital warts, paroxysmal nocturnal hemoglobinuria, multiple sclerosis, skin lesions, surface wounds, eye infections, HIV AIDS, condyloma acuminatum, severe blood loss, hypervolemia, hypoproteinemia, adult and juvenile rheumatoid arthritis, diagnosis of pancreatic exocrine dysfunction and gastrinoma, prophylactic use to reduce perioperative blood loss and the need for blood transfusion, cystic fibrosis, chronic pancreatitis, pancreatic duct blockage, severe hypoglycemia, gastrointestinal imaging, heparin-induced thrombocytopenia, prevention of HIV-induced weight loss, post-menopausal osteoporosis, rehydration, screening for adrenocortical insufficiency, chronic plaque psoriasis, hemophilia, cervical dystonia, acute evolving transmural myocardial infarction, pulmonary embolism, deep vein thrombosis, arterial thrombosis or embolism, occlusion of arteriovenous cannulae, primary insulin-like growth factor deficiency, chronic dermal ulcers, severe skin burns, vaccine adjuvant, diabetes (type I and II), obesity, metabolic syndrome X, coronary artery thrombosis, IV catheter clearance, Fabry's disease, cervical dystonia, severe primary axillary hyperhidrosis, strabismus, blepharospasm, increasing reduced platelet levels due to chemotherapy, skin and skin structure infections, bone marrow transplant, hemorrhagic complications in hemophilia A and B, Gaucher's disease, increasing leukocyte production, neutropenia, mucopolysaccharidosis VI, diagnosing extrahepatic malignant cancers, imaging colorectal tumors, acromegaly, anemia, von Willebrand disease, Factor XIII deficiency, mucositis (mouth sores), female infertility, panacinar emphysema, and dwarfism.

In another embodiment, the medical conditions include, but are not limited to, acromegaly, carcinoid tumors, vasoactive intestinal peptide tumors, osteoporosis, ovarian cancer, breast cancer, non-small cell lung cancer, pancreatic cancer, skin and structure infections, staphylococcus aureus bloodstream infections, chronic lymphocytic leukemia, indolent B-cell non-Hodgkin's lymphoma, vitamin B 12 deficiencies (e.g., vegetarians, malabsorption, low intrinsic factor, bacterial or parasitic infection), multiple sclerosis, multiple myeloma, mantle cell lymphoma, growth hormone deficiencies, Prader-Willi Syndrome (PWS), Turner Syndrome, idiopathic short stature and a combination thereof.

The present invention will now be described in more detail with reference to the following examples. However, these examples are given for the purpose of illustration and are not to be construed as limiting the scope of the invention

### EXAMPLES

### Example 1

### Study of Bioavailability for Tablets Prepared by Wet Granulation Versus Dry Granulation

a. Preparation of Tablets by Wet Granulation

The formulation of the tablets prepared by wet granulation is provided in Table 1-a. The tablet was prepared as follows: A dry powder mixture of sodium caprate, mono sodium alendronate trihydrate, and PVP K30 was granulated using a 25 percentage solution. The granulate was then screened and subsequently fluid bed dried and milled. Then, granulates were blended with aerosol, mannitol, polyplasdone, and stearic acid. The blended mixture was compressed and subcoated. Finally, the mixture was enteric coated.

The investigators of the present invention have attempted to prepare tablets including 20 mg alendronate and 550 mg C10 using wet granulation. However, the tablets failed the disintegration test due to unacceptable friability and coating properties. It is observed that the maximum amount of C10 included in a tablet prepared by wet granulation is about 250 mg to 300 mg per tablet for the tablet to possess acceptable coating and friability properties.

Please note: in examples 1 and 2. All tablets were prepared using alendronate monosodium salt trihydrate. In tables 1(a) and 1(b), the amount of alendronic acid is the molar equivalent of the alendronate monosodium salt trihydrate (7.86 mg alendronate monosodium salt trihydrate is molar equivalent of 6.0 mg free acid, alendronic acid). In all Examples and Figures contained herein referencing sodium alendronate or alendronic acid tablets, compositions contain sodium alendronate, and quantities are expressed as the molar equivalent amount of alendronic acid.

b. Preparation of Tablets by Dry Granulation

The formulation of the tablets prepared by dry granulation is provided in Table 1-b. The tablet was prepared as follows: sodium caprate and sorbitol (about 293 mg of Parteck SI 400) were firstly dry mixed. Then, a slugging process was performed on the dry mixture. Then, the mixture was initially comminuted and milled. The mixture was blended with excipients and then compressed and sub coated. Finally, the mixture was enteric coated. During the preparation, the investigators discovered that when the sodium caprate is dry granulated, at least 550 mg sodium caprate may be incorporated into one tablet. It is unexpected that dry granulation produces a more compact material than wet granulation.

c. Comparison of the Bioavailability Data

The bioavailability of tablets of alendronic acid prepared by dry granulation was compared with those prepared by wet granulation. As illustrated in Tables 1-a and 1-b, the bioavailability (% dose excreted in urine) of the tablet including 550 mg sodium caprate prepared by dry granulation was significantly improved compared to two tablets including total 500 mg sodium caprate prepared by wet granulation. The investigators of the present invention believe that the improved bioavailability is due to the ability to incorporate more sodium caprate in one tablet prepared using dry granulation and the more rapid dissolution afforded by the tablets prepared by dry granulation as discussed below in Example 2.

TABLE 1(a)
The formulation, bioavailability, dosing condition, etc of the tablets prepared by wet granulation

| | Cumulative | | | | | |
|---|---|---|---|---|---|---|
| | amount | % Dose | | | | |
| No. of | excreted | excreted | | | | |
| Tablet | (mg) | in urine | Formulation | Dosing | Friability | Disintegration |
| A | 0.09 ± 0.03 | 0.26% | I tablet Fosamax ® 35 mg | Fasted | | |
| | CV % = 33.6 | | | 1 tablet | | |
| B | 0.26 ± 0.11 | 1.49% | PD16538 Fasted | Fasted | PD 16627 | Conditions: 1 hour |
| | CV % = 40.5 | | Each tablet (11 mm) (2 dosed) | 2 tablets | Uncoated | simulated gastric fluid |
| | | | Alendronic acid 8.75 mg | | 0.1% | (SGF) without enzymes |
| | | | Sodium Caprate 250.0 mg | | | (observe for disintegration). |
| | | | Tablet weight 0.553 g | | | Phosphate buffer pH 6.8 |
| | | | Total dose = 17.5 mg alendronate and | | | (record time taken to |
| | | | 500 mg sodium caprate | | | dissolve) |
| C | 0.03 ± 0.03 | 0.17% | PD16538 | Fasted | | PD16538 Enteric coated |
| | CV % = 109.8 | | Each tablet (11 mm) (2 dosed) | 2 tablets | | SGF: No evidence of |
| | | | Alendronic acid 8.75 mg | | | disintegration, cracking or |
| | | | Sodium Caprate 250.0 mg | | | softening |
| | | | Tablet weight 0.553 g | | | Phosphate buffer pH 6.8: |
| | | | Total dose = 17.5 mg sodium alendronate | | | Dissolved within 20 minutes |
| | | | and 500 mg sodium caprate | | | |
| D | 0.28 ± 0.30 | 1.6% | PD16531 Fasted | Fasted | PD 16677 | PD16531 |
| | CV % = 106.1 | | Each tablet (9 mm) (2 dosed) | 2 tablets | Uncoated | Enteric coated |
| | | | Alendronic acid 8.75 mg | | 0.2% | As above |
| | | | Sodium Caprate 125 mg | | | |
| | | | Tablet weight: 0.289 g | | | |
| | | | Total dose = 17.5 mg sodium alendronate | | | |
| | | | and 250 mg sodium caprate | | | |
| E | 0.20 ± 0.16 | 1.14% | PD16540 Fasted | Fasted | PD 16678 | PD16540 |
| | CV % = 79.0 | | 1 tablet dosed (11 mm) | 1 Tablet | Uncoated | Enteric coated as above |
| | | | Alendronic acid 17.5 mg | | 0.2% | |
| | | | Sodium Caprate 250 mg | | | |
| | | | PVP 35.425 mg | | | |
| | | | Tablet weigh: 0.577 g | | | |

TABLE 1(b)
The formulation, bioavailability, dosing condition, etc of the tablets prepared by dry granulation

| | Cumulative | % | | | | |
|---|---|---|---|---|---|---|
| | amount | Dose | | | | |
| No. of | excreted | excreted | | | Mean | |
| Tablet | (mg) | in urine | Formulation | Dosing | Friability | Disintegration |
| A | 0.11301 ± 0.05 | 0.323% | Fosamax 35 mg | overnight fast, upright for 4 | Uncoated = | |
| | 488 | | | hours after dosing | 0.71% | |
| | CV % = 48.6 | | | | | |
| B | 0.20312 ± .087 | 3.39% | Alendronic acid 6.0 mg | dosed as above | As above | 0.01N HCl: pass (no effect |
| | 17 | | Sodium caprate 550 mg | | | on tablets) |
| | CV % = 42.9 | | Tablet Core weight = 900 mg | | | Phosphate pH 6.8 = |
| | | | enteric tablet weight = 1035.09 mg | | | Disintegration in approx. |
| C | 0.22035 ± 0.16 | 3.67% | Alendronic acid 6.0 mg | Dosed at 10:30 pm | | 17 min 30 sec |
| | 313 | | Sodium caprate 550 mg | following 6 pm meal) | | |
| | CV % = 74 | | Tablet Core weight = 900 mg | fasting from 6.30 pm until | | |
| | | | enteric tablet weight = 1035.09 mg | breakfast. Laid down for 2 | | |
| | | | | hours after dosing. | | |
| D | 0.03299 ± 0.05 | 0.55% | Alendronic acid 6.0 mg | Dosed in the AM with the | As above | As above |
| | 372 | | Sodium caprate 550 mg | standard FDA high fat | | |
| | CV % = 162.9 | | Tablet Core weight = 900 mg | breakfast, upright position | | |
| | | | enteric tablet weight = 1035.09 mg | for 4 hours after dosing. | | |

TABLE 2
The dissolution rate and amount of alendronate and C10
8.75 mg alendronic acid; 250 mg C10 per tablet

| | | | | Amount |
|---|---|---|---|---|
| | % C10 | % Alendronic | Amount C10 | Alendronic acid |
| | Released | acid Released | Released (mg) | Released (mg) |
| 0 | 0 | 0 | 0 | 0 |
| 10 | 8.5 | 9.4 | 21.25 | 0.8225 |
| 20 | 46.8 | 47.1 | 117 | 4.12125 |
| 30 | 78.3 | 79.7 | 195.75 | 6.97375 |
| 45 | 98.8 | 97.5 | 247 | 8.53125 |
| 60 | 99.3 | 98.7 | 248.25 | 8.63625 |

8.75 mg alendronic acid 125 mg C10 per tablet

| | | | | Amount |
|---|---|---|---|---|
| | % C10 | % alendronic acid | Amount C10 | alendronic acid |
| | Released | Released | Released (mg) | Released (mg) |
| 0 | 0 | | 0 | 0 |
| 10 | 6 | 11.8 | 7.5 | 1.0325 |
| 20 | 57.1 | 54.1 | 71.375 | 4.73375 |
| 30 | 89.3 | 86.9 | 111.625 | 7.60375 |
| 45 | 101.6 | 98.8 | 127 | 8.645 |
| 60 | 102 | 99.6 | 127.5 | 8.715 |

17.5 mg alendronic acid; 250 mg C10 per tablet

| | | | | Amount |
|---|---|---|---|---|
| | % C10 | % alendronic acid | Amount C10 | alendronic acid |
| | Released | Released | Released (mg) | Released (mg) |
| 0 | 0 | 0 | 0 | 0 |
| 10 | 13.3 | 7.8 | 33.25 | 1.365 |
| 20 | 51.2 | 46.3 | 128 | 8.1025 |
| 30 | 80.1 | 76.7 | 200.25 | 13.4225 |
| 45 | 101.9 | 97.9 | 254.75 | 17.1325 |
| 60 | 102.4 | 99.9 | 256 | 17.4825 |

6.00 mg alendronic acid; 550 mg C10 per tablet

| | | | | Amount |
|---|---|---|---|---|
| | | | | Alendronic acid |
| | % C10 | % alendronic acid | Amount C10 | Released (mg) |
| | Released | Released | Released (mg) | *EXTRAPOLATED |
| 0 | 0 | N/A | 0 | 0 |
| 10 | 5.45 | N/A | 29.975 | 0.327* |
| 20 | 65.25 | N/A | 358.875 | 3.915* |
| 30 | 92.57 | N/A | 509.135 | 5.5542* |
| 45 | 96.67 | N/A | 531.685 | 5.8002* |
| 60 | N/A | N/A | N/A | N/A |

*Extrapolated amount is predicted based on the assumption that the enhancer and the active ingredient (e.g., sodium alendronate) is released at substantially the same rate.

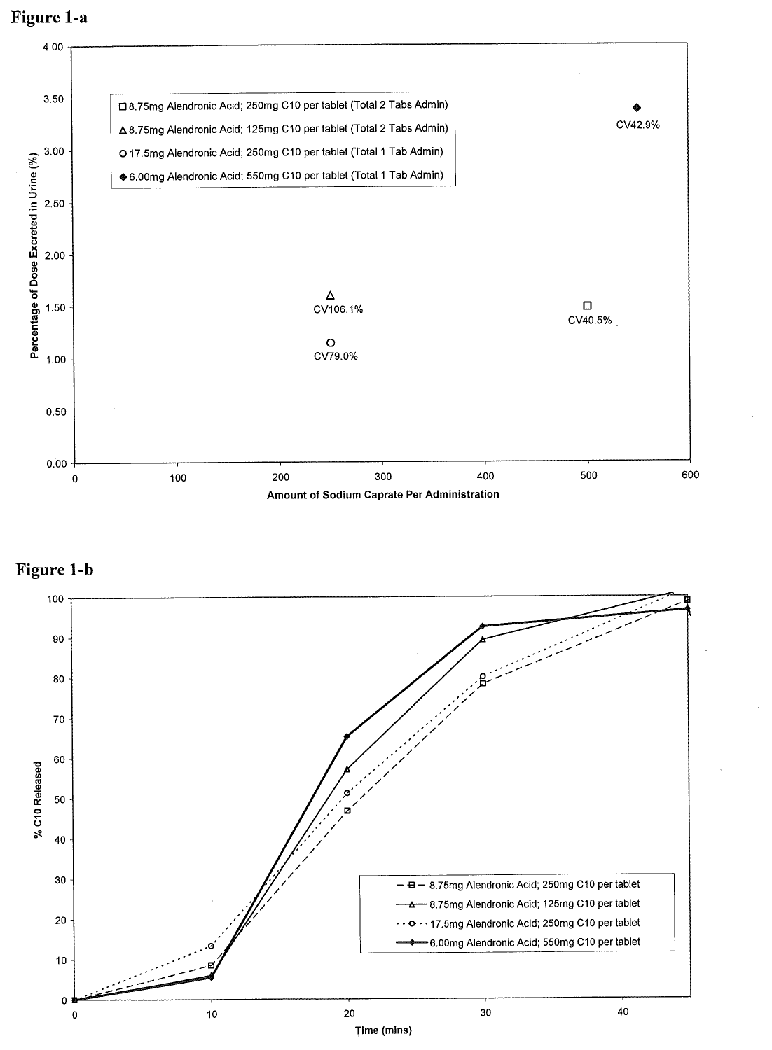

FIG. 1-a graphically demonstrates the bioavailability for the various formulations prepared by using wet granulation versus the formulation prepared by dry granulation. The tablets prepared by dry granulation are shown as square, triangle and round shapes. The tablets prepared by wet granulation are shown as diamond shape. FIG. 1 shows that the bioavailability for tablets prepared by wet granulation is similar, regardless of the amount of sodium caprate dosed. The bioavailability for tablets prepared by the dry granulation (diamond) is approximately double compared to tablets with similar formulation, but prepared by wet granulation (square).

As shown in FIG. 1-a, the tablet manufactured by a dry granulation (diamond) achieves the highest percentage of total dose excreted in urine. Therefore, dry granulation is the preferred manufacturing technique for enhancing absorption via administration of water soluble enhancers, as evidenced by these data collected using medium chain fatty acid salts. Moreover, it has been observed that the bioavailability of two tablets including a total of 500 mg C10 (square) was similar to one tablet including 250 mg C10 (circle) and much lower than one tablet including 550 mg C10 (diamond). Thus, the amount of enhancer in the tablets does not appear to be the primary variable affecting the bioavailability of the tablets. It is further indicated that the required amount of C10 is preferably included in a single dosage unit rather than multiple dosage units.

FIGS. 1-b and 1-c graphically show the dissolution profile of C10 for tablets containing different amount of C10, FIG. 1-b demonstrates dissolution profiles of C10 in phosphate buffer pH 6.8, which is expressed as % released C10 per tablet. FIG. 1-c demonstrates dissolution profiles of C10 in phosphate buffer pH 6.8, which is expressed as the amount of released C10 per tablet. The dissolution test was carried out on uncoated tablets. The tablets were placed in about 900 ml of pH 6.8 phosphate buffer and stirred at 50 rpm using the USP Paddle Apparatus. The system was maintained at 37° C. A sample was taken at prescribed time points to generate dissolution profiles for alendronic acid and C10. As shown in both FIGS. 1-b and 1-c, after about 20 minutes, the tablet containing about 550 mg C10 has a relatively better dissolution profile.

FIG. 1-d graphically shows the relationship between in vivo performance (% alendronic acid excreted in urine) and in vitro performance (Amount of alendronic acid released at T=20 minutes in Phosphate buffer pH 6.8 (USP Paddle Apparatus, 50 rpm, 37° C., 900 mL, 2 hrs in 0.1N HCl). FIG. 1-e demonstrates the relationship between in vivo performance (% alendronic acid excreted in urine) and in vitro performance (Amount of C10 released at T=20 minutes in Phosphate buffer pH 6.8 (USP Paddle Apparatus, 50 rpm, 37° C., 900 mL, 2 hrs in 0.1N HCl) and shows that the dry granulation tablet has much better in vivo absorption. As shown in FIG. 1-d, there is no apparent correlation between an increased amount of alendronic acid dissolved in vitro and the observed increased in vivo performance of the tablet containing about 550 mg C10. However, as shown in FIG. 1-e, there is a correlation between an increased amount of C10 dissolved in vitro (per dosage form) and the increased in vivo performance of the tablet containing about 550 mg C10. Therefore, the increased amount of C10 per dosage form provides a faster dissolution rate of C10 and then the faster dissolution rate leads to an improved bioavailability of the tablets.

### Example 3

### Disintegration Time of Tablets Including Different Excipients

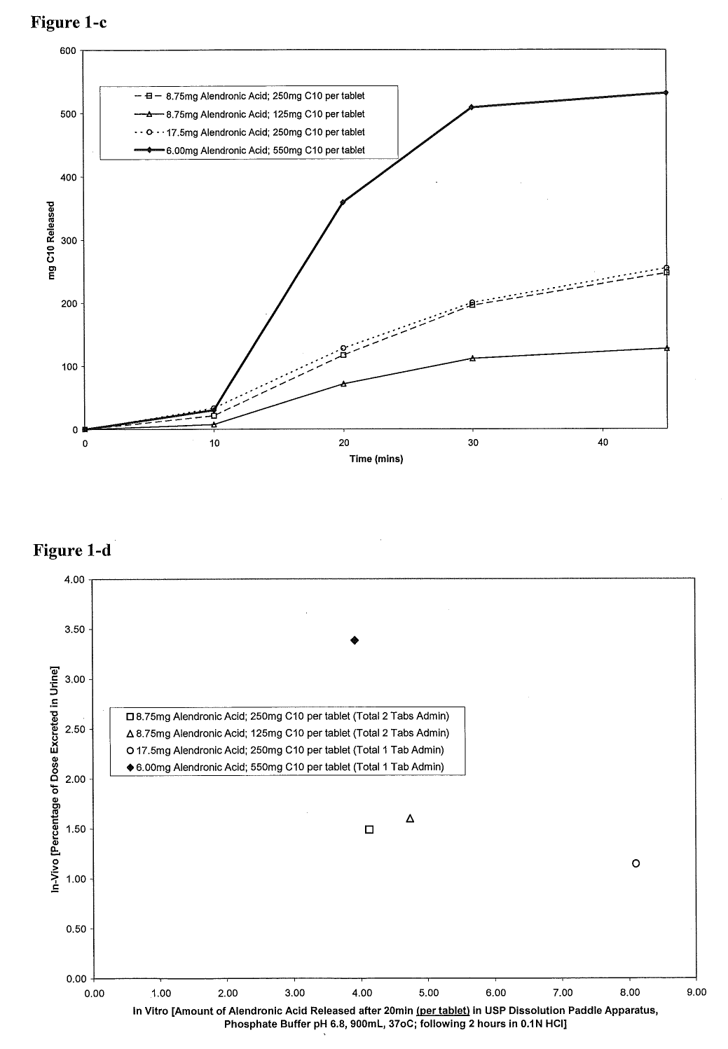

A study of disintegration time of tablets containing a water soluble bioavailability enhancer and different excipients was carried out. The results are summarized in FIG. 2. Microcrystalline cellulose and pregelatinized starch are widely used in pharmaceuticals for their tablet diluent and disintegration properties. Saccharides are widely used in pharmaceutical formulations as a diluent but are not known to have disintegration properties. The formulae of tablets used in EXP 1366, EXP 1371, EXP 1372, and EXP 1373 are provided in Tables 3-6. As shown in FIG. 2, the formulations including saccharides (e.g., sorbitol or mannitol) disintegrate significantly faster than formulations without saccharides. It is concluded that, when incorporated with effective amount of water soluble bioavailability enhancers, tablets made with saccharides disintegrate more quickly. It is surprising that the enhancer formulations made with binders with disintegration properties disintegrate slower than enhancer formulations with saccharides.

TABLE 3
Formulation of tablets used in EXP 1366

| | Composition | Composition |
|---|---|---|
| Ingredient name | % w/w | mg/tab |
| Sodium Caprate | 78.17 | 550.0 |
| Sorbitol Parteck | 21.32 | 150.0 |
| SI 150 | | |
| Stearic acid | 0.51 | 3.6 |

TABLE 4
Formulation of tablets used in EXP 1371

| | Composition | Composition |
|---|---|---|
| Ingredient name | % w/w | mg/tab |
| Sodium Caprate | 78.17 | 550.0 |
| Mannitol | 21.32 | 150.0 |
| Pearlitol 100SD | | |
| Stearic acid | 0.51 | 3.6 |

TABLE 5
Formulation of tablets used in EXP 1372

| | Composition | Composition |
|---|---|---|
| Ingredient name | % w/w | mg/tab |
| Sodium Caprate | 78.17 | 550.0 |
| Microcrystalline | 21.32 | 150.0 |
| cellulose Avicel | | |
| PH-102 | | |
| Stearic acid | 0.51 | 3.6 |

TABLE 6
Formulation of tablets used in EXP 1373

| | Composition | Composition |
|---|---|---|
| Ingredient name | % w/w | mg/tab |
| Sodium Caprate | 78.17 | 550.0 |
| Starch 1500 | 21.32 | 150.0 |
| Stearic acid | 0.51 | 3.6 |

### Example 4

### Dissolution Rate of Tablets Including Sorbitol Versus Tablets Including Microcrystalline Cellulose for Tablets Including Zoledronic Acid and C10

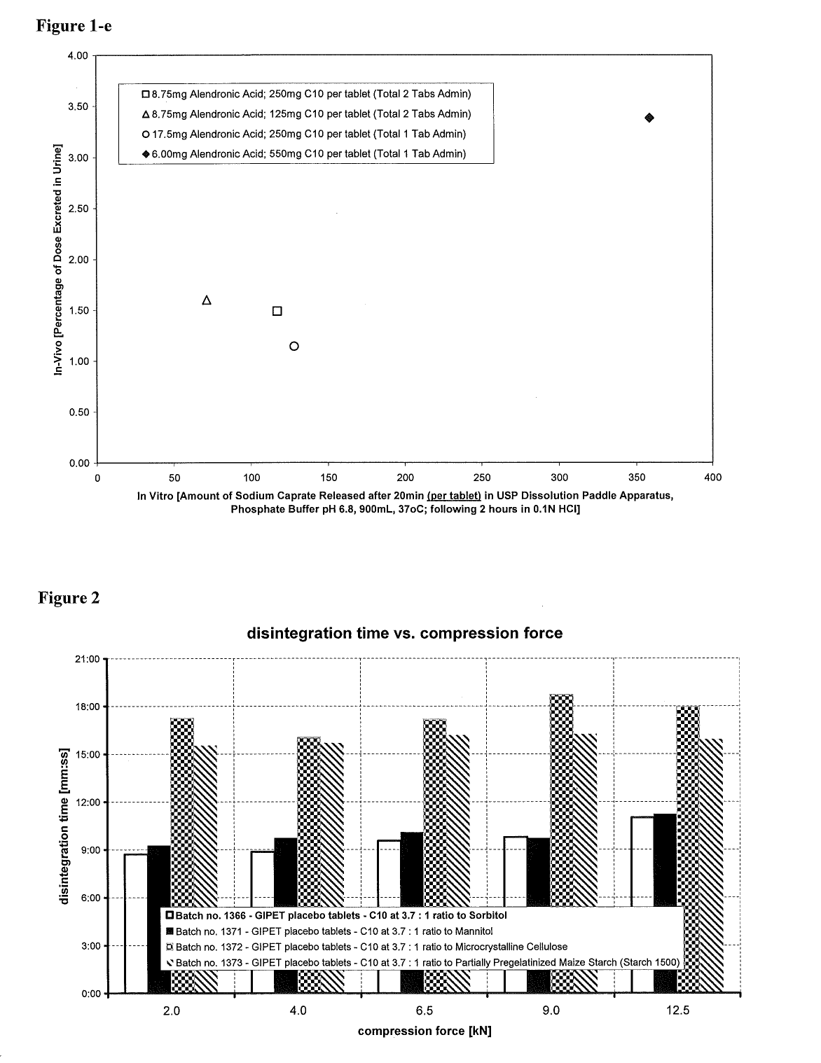

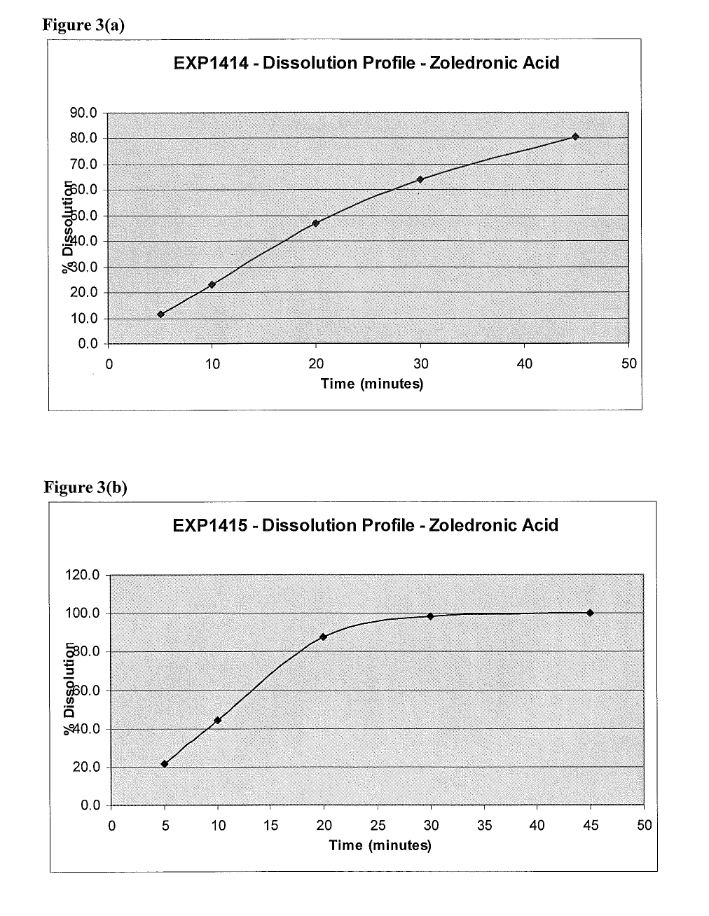

A study for testing the dissolution rate of zoledronic acid tablets containing a water soluble enhancer made with sorbitol versus tablets made with microcrystalline cellulose was carried out. The formulation of tablets including microcrystalline cellulose (EXP 1414) and tablets including sorbitol (EXP 1415) is provided in Tables 7 and 8 respectively. For both EXP 1414 and 1415, there was no coating on the tablets. The dissolution of zoledronic acid and C10 for EXP 1414 and 1415 is shown in Tables 9-12. The dissolution profile for zoledronic acid and C10 is graphically illustrated in FIGS. 3 and 4.

As shown in FIGS. 3 and 4 and Tables 9-12, the dissolution for zoledronic acid and C10 in EXP 1415 (tablets including sorbitol) is significantly faster compared to those in EXP 1414 (tablets including microcrystalline cellulose). For example, C10 in EXP 1415 has a dissolution of about 100% in about 30 minutes. Zoledronic acid in EXP 1415 has a dissolution of about 100% in about 30 minutes. In contrast, the dissolution of C10 and zoledronic acid in EXP 1414 only reaches about 80% after 45 minutes. Therefore, it may be concluded that the dissolution rate of zoledronic acid and C10 is significantly improved in the presence of sorbitol.

In addition, comparing FIG. 3(b) with FIG. 4(b), the dissolution of zoledronic acid and C10 is substantially similar for tablets in EXP 1415. For example, zoledronic acid in EXP 1415 has a dissolution of about 100% in about 30 minutes, C10 in EXP 1415 has a dissolution of about 100% in about 30 minutes, as well. In contrast, the dissolution of zoledronic acid and C10 in EXP 1414 is not substantially similar. This result was surprising as well, and is perhaps due to the unexpected slower disintegration times observed with tablets comprising microcrystalline cellulose.

TABLE 7
Formulation of tablets used in Exp 1414

| | | Composition |
|---|---|---|
| Excipient name/API | Composition % (w/w) | mg/tab |
| C10 granules | 76.01 | 550.00 |
| Microcrystalline | 20.73 | 150.00 |
| cellulose | | |
| Stearic acid | 0.50 | 3.60 |
| Zoledronic acid | 2.76 | 20.00 |
| Final tablet weight | 100 | 723.60 |

TABLE 8
Formulation of tablets used in Exp 1415

| | Composition % | Composition |
|---|---|---|
| Excipient name/API | (w/w) | mg/tab |
| C10 granules | 76.01 | 550.00 |
| Sorbitol Parteck SI 150 | 20.73 | 150.00 |
| Stearic acid | 0.50 | 3.60 |
| Zoledronic acid | 2.76 | 20.00 |
| Final tablet weight | 100 | 723.60 |

TABLE 9
Dissolution rate of Zoledronic acid of tablets in EXP 1414
% Dissolution

| | Sampling time points (minutes) | | | | |
|---|---|---|---|---|---|
| | 5 | 10 | 20 | 30 | 45 |
| Vessel 1 | 11.1 | 21.7 | 43.7 | 62.6 | 86.3 |
| Vessel 2 | 12.7 | 26.9 | 59.9 | 78.6 | 91.1 |
| Vessel 3 | 10.8 | 20.7 | 37.5 | 50.6 | 64.3 |
| Mean: | 11.5 | 23.1 | 47.0 | 63.9 | 80.5 |
| % CV: | 8.9 | 14.6 | 24.6 | 21.9 | 17.7 |

TABLE 10
Dissolution rate of zoledronic acid of tablets in EXP 1415
% Dissolution

| | Sampling time points (minutes) | | | | |
|---|---|---|---|---|---|
| | 5 | 10 | 20 | 30 | 45 |
| Vessel 1 | 26.5 | 54.6 | 98.1 | 105.2 | 87.8 |
| Vessel 2 | 15.8 | 44.4 | 67.6 | 81.6 | 104.5 |
| Vessel 3 | 21.7 | 34.7 | 97.8 | 107.8 | 107.7 |
| Mean: | 21.3 | 44.6 | 87.8 | 98.2 | 100.0 |
| % CV: | 25.3 | 22.3 | 19.9 | 14.7 | 10.7 |

TABLE 11
Dissolution rate of C10 in tablets in EXP 1414
% Dissolution

| | Sampling time points (minutes) | | | | |
|---|---|---|---|---|---|
| | 5 | 10 | 20 | 30 | 45 |
| Vessel 1 | 23.1 | 41.8 | 60.8 | 72.4 | 84.9 |
| Vessel 2 | 30.2 | 45.7 | 70.7 | 81.3 | 86.0 |
| Vessel 3 | 26.2 | 43.6 | 62.3 | 73.2 | 80.1 |
| Mean: | 26.5 | 43.7 | 64.6 | 75.6 | 83.6 |
| % CV: | 13.4 | 4.6 | 8.3 | 6.5 | 3.7 |

TABLE 12
Dissolution rate of C10 in tablets in EXP 1415
% Dissolution

| | Sampling time points (minutes) | | | | |
|---|---|---|---|---|---|
| | 5 | 10 | 20 | 30 | 45 |
| Vessel 1 | 36.4 | 63.4 | 95.9 | 101.3 | 91.9 |
| Vessel 2 | 38.0 | 58.2 | 87.7 | 93.5 | 97.5 |
| Vessel 3 | 32.3 | 62.8 | 92.8 | 100.0 | 98.6 |
| Mean: | 35.6 | 61.5 | 92.1 | 98.3 | 96.0 |
| % CV: | 8.3 | 4.6 | 4.5 | 4.2 | 3.8 |

### Example 5

### F1 and F2 Study of Tablets Including Sorbitol Versus Microcrystalline Cellulose

F1 (difference factor) and f2 (similarity factor) analysis has also been conducted to analyze the dissolution profiles of the formulations described herein. The testing and calculation of f1 and f2 are known to one of skill in the art (See e.g., J. W. Moore and H. H. Flanner, Mathematical Comparison of curves with an emphasis on in vitro dissolution profiles. Pharm. Tech. 20(6): 64-74, 1996; V. P. Shah, etc., In vitro dissolution profile comparison-statistics and analysis of the similarity factor, f2. Pharm. Res. 15: 889-896, 1998.)

### (1) Tablets Including Zoledronic Acid and C10

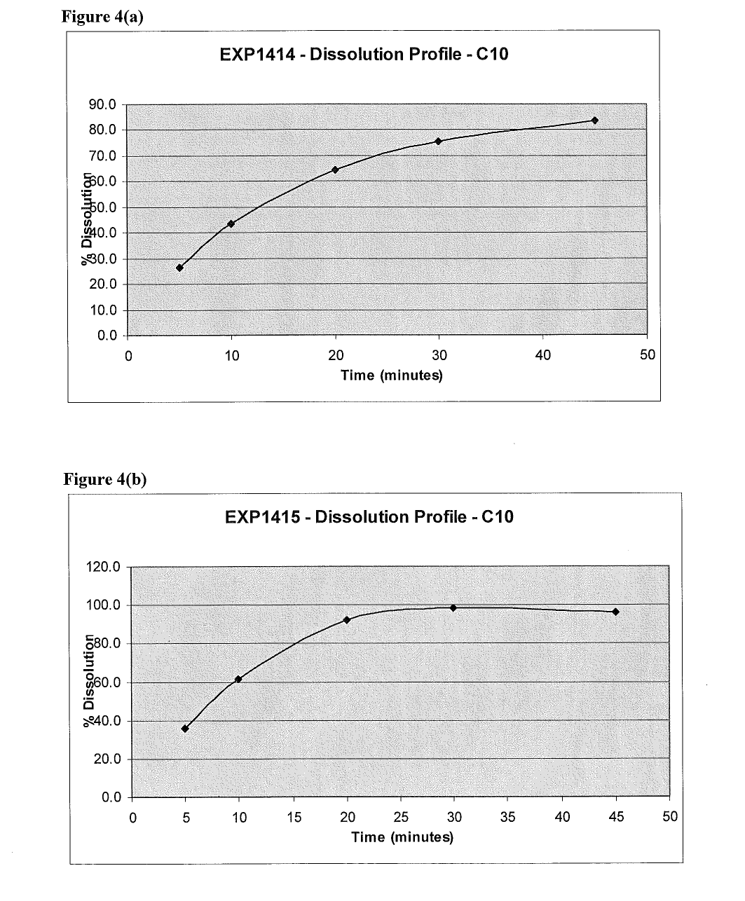

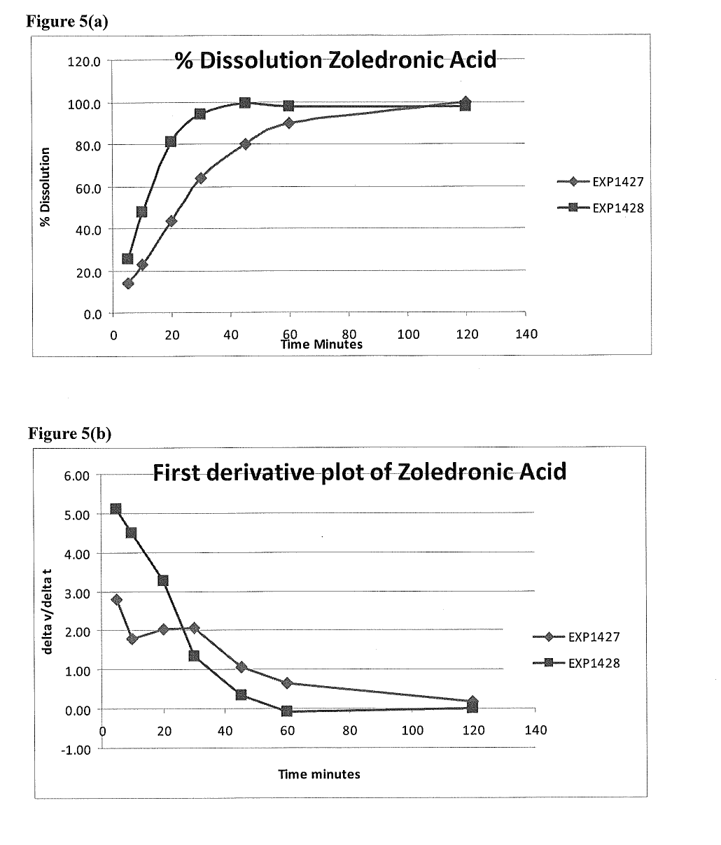

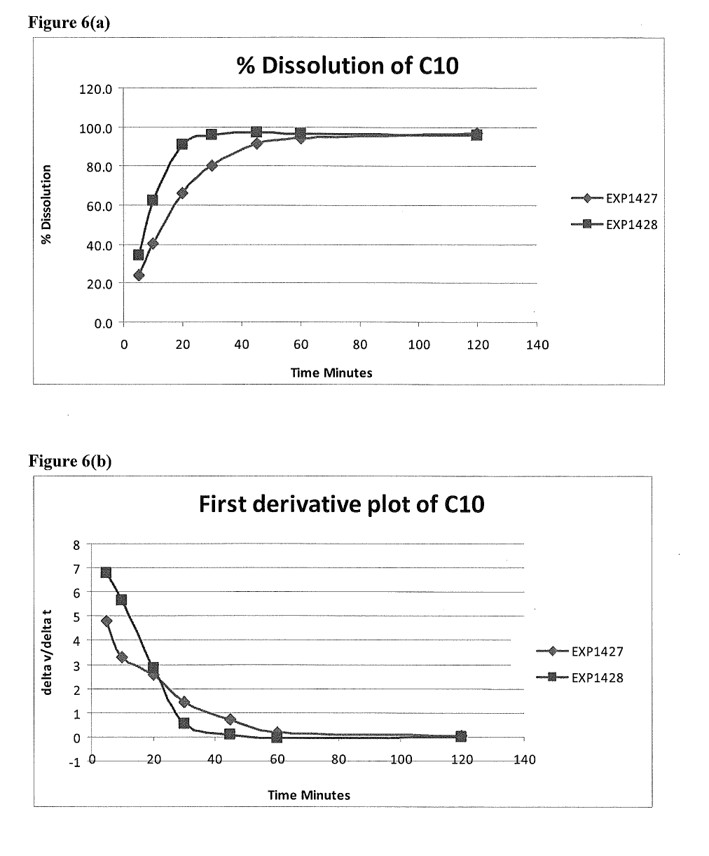

The formulation of tablets used in EXP 1427 is the same as the tablets used in EXP 1414 (tablets including microcrystalline cellulose). The formulation of tablets used in EXP 1428 is the same as the tablets used in EXP 1415 (tablets including sorbitol). The dissolution data and f1 and 12 analysis for EXP 1427 and 1428 are provided in Tables 13-23. The dissolution profile and first derivative analysis are graphically described in FIGS. 5 to 7. As shown from Tables 13-20 and FIGS. 5 to 7, the f1 and f2 analysis demonstrate that the dissolution profiles of zoledronic acid and C10 in the tablets of EXP 1428 (including sorbitol) are substantially similar. However, the dissolution profiles of zoledronic acid and C10 in the tablets of EXP 1427 are distinctly different. This observation indicates that the presence of sorbitol in the formulation may provide substantially similar dissolution rate of the active ingredient (zoledronic acid) and the enhancer (C10).

TABLE 13
Dissolution rate of zoledronic acid in tablets in EXP 1427
% Dissolution

| | Sampling time points (minutes) | | | | | | |
|---|---|---|---|---|---|---|---|
| | 5 | 10 | 20 | 30 | 45 | 60 | 120 |
| Vessel 1 | 14.4 | 23.2 | 38.0 | 54.0 | 80.8 | 93.5 | 95.0 |
| Vessel 2 | 19.8 | 28.3 | 45.4 | 61.3 | 84.9 | 105.5 | 105.9 |
| Vessel 3 | 11.4 | 21.8 | 49.2 | 79.8 | 92.6 | 97.3 | 102.0 |
| Vessel 4 | 9.2 | 18.0 | 37.6 | 52.9 | 57.0 | 61.5 | 96.0 |
| Vessel 5 | 18.5 | 28.5 | 49.2 | 63.9 | 78.6 | 83.7 | 98.6 |
| Vessel 6 | 11.1 | 17.9 | 40.8 | 72.5 | 87.1 | 98.8 | 99.8 |
| Mean: | 14.0 | 23.0 | 43.4 | 64.1 | 80.2 | 90.0 | 99.5 |
| % CV: | 30.6 | 20.6 | 12.2 | 16.4 | 15.4 | 17.5 | 4.0 |

TABLE 14
Dissolution rate of C10 in tablets in EXP 1427
% Dissolution

| | Sampling time points (minutes) | | | | | | |
|---|---|---|---|---|---|---|---|
| | 5 | 10 | 20 | 30 | 45 | 60 | 120 |
| Vessel 1 | 18.3 | 31.1 | 51.8 | 68.8 | 89.5 | 97.1 | 97.4 |
| Vessel 2 | 18.1 | 33.7 | 58.9 | 76.2 | 92.9 | 96.8 | 96.0 |
| Vessel 3 | 24.9 | 43.3 | 71.5 | 88.2 | 95.2 | 95.6 | 96.8 |
| Vessel 4 | 32.9 | 51.6 | 77.2 | 84.7 | 85.8 | 86.5 | 97.1 |
| Vessel 5 | 25.6 | 43.7 | 70.8 | 81.7 | 87.3 | 90.6 | 97.4 |
| Vessel 6 | 23.7 | 38.5 | 66.3 | 82.7 | 97.8 | 98.9 | 97.1 |
| Mean: | 23.9 | 40.3 | 66.1 | 80.4 | 91.4 | 94.2 | 97.0 |
| % CV: | 22.9 | 18.5 | 14.0 | 8.6 | 5.1 | 5.0 | 0.5 |

TABLE 15
Dissolution rate of zoledronic acid in tablets in EXP 1428
% Dissolution

| | Sampling time points (minutes) | | | | | | |
|---|---|---|---|---|---|---|---|
| | 5 | 10 | 20 | 30 | 45 | 60 | 120 |
| Vessel 1 | 24.7 | 39.5 | 62.6 | 76.7 | 96.2 | 94.2 | 94.7 |
| Vessel 2 | 27.0 | 54.6 | 89.7 | 97.3 | 97.0 | 96.1 | 95.5 |
| Vessel 3 | 27.6 | 55.7 | 91.8 | 101.6 | 100.8 | 99.8 | 99.0 |
| Vessel 4 | 29.2 | 49.8 | 86.1 | 103.1 | 109.1 | 107.6 | 107.9 |
| Vessel 5 | 23.2 | 45.1 | 76.1 | 90.3 | 93.4 | 92.7 | 91.9 |
| Vessel 6 | 21.4 | 43.2 | 78.2 | 95.7 | 97.9 | 97.1 | 97.5 |
| Mean: | 25.5 | 48.0 | 80.8 | 94.1 | 99.1 | 97.9 | 97.8 |
| % CV: | 11.5 | 13.5 | 13.4 | 10.3 | 5.5 | 5.5 | 5.7 |

TABLE 16
Dissolution rate of C10 in tablets in EXP 1428
% Dissolution

| | Sampling time points (minutes) | | | | | | |
|---|---|---|---|---|---|---|---|
| | 5 | 10 | 20 | 30 | 45 | 60 | 120 |
| Vessel 1 | 26.1 | 49.3 | 80.5 | 93.8 | 97.5 | 97.1 | 95.7 |
| Vessel 2 | 34.0 | 64.6 | 94.4 | 96.3 | 95.0 | 95.7 | 95.1 |
| Vessel 3 | 33.4 | 61.6 | 92.5 | 96.4 | 97.5 | 95.1 | 95.2 |
| Vessel 4 | 29.0 | 56.5 | 91.4 | 95.2 | 97.5 | 96.6 | 95.8 |
| Vessel 5 | 41.5 | 69.8 | 91.8 | 97.1 | 97.2 | 96.8 | 96.7 |
| Vessel 6 | 40.3 | 72.0 | 92.9 | 96.6 | 97.4 | 96.0 | 96.7 |
| Mean: | 34.0 | 62.3 | 90.6 | 95.9 | 97.0 | 96.2 | 95.9 |
| % CV: | 17.8 | 13.6 | 5.6 | 1.2 | 1.0 | 0.8 | 0.7 |

TABLE 17
F1 analysis of zoledronic acid and C10 for EXP 1428

| | EXP1428 | EXP1428 | | | |
|---|---|---|---|---|---|
| | API | C10 | | | |
| Time | Tₜ | Rₜ | Rₜ − Tₜ | | |
| 5 | 25.5 | 34.0 | 8.5 | | |
| 10 | 48.0 | 62.3 | 14.3 | | |
| 20 | 80.8 | 90.6 | 9.8 | | |
| 30 | 94.1 | 95.9 | 1.8 | | |
| 45 | 99.1 | 97.0 | 2.1 | | |
| 60 | 97.9 | 96.2 | 1.7 | | |
| | | | | SUM(Rₜ − Tₜ) | 34.4 |
| | F1: | 12.2 | | SUM(Rₜ) | 282.8 |
| | F2: | 50.6 | | SUM(Rₜ − | 0.12164074 |
| | | | | Tₜ)/SUM(Rₜ) | |
| | | | | F1 = | 12.1640736 |

TABLE 18
F2 analysis of zoledronic acid and C10 for EXP 1428

| | EXP1428 | EXP1428 | | |
|---|---|---|---|---|
| | API | C10 | | |
| Time | Tₜ | Rₜ | Rₜ − Tₜ | (Rₜ − Tₜ)² |
| 5 | 25.5 | 34.0 | 8.5 | 72.25 |
| 10 | 48.0 | 62.3 | 14.3 | 204.49 |
| 20 | 80.8 | 90.6 | 9.8 | 96.04 |
| 30 | 94.1 | 95.9 | 1.8 | 3.24 |
| 45 | 99.1 | 97.0 | 2.1 | 4.41 |
| 60 | 97.9 | 96.2 | 1.7 | 2.89 |
| | | | SUM(Rₜ − Tₜ)² | 376.02 |
| | | | N = | 4 |
| | | | 1/N [SUM(Rₜ − Tₜ)²] | 94.005 |
| | | | 1 + 1/N [SUM(Rₜ − Tₜ)²] | 95.005 |
| | | | {1 + 1/N [SUM(Rₜ − Tₜ)²]}^{-0.5} | 0.102595135 |
| | | | {1 + 1/N [SUM(Rₜ − Tₜ)²]}^{-0.5} * 100 | 10.25951354 |
| | | | Log{{1 + 1/N [SUM(Rₜ − Tₜ)²]}^{-0.5} * 100} | 1.011126769 |
| | | | F2 (50 * Log{{1 + 1/N [SUM(Rₜ − Tₜ)²]}^{-0.5} * | 50.55633844 |
| | | | 100} | |

TABLE 19
F1 analysis of zoledronic acid and C10 for EXP 1427

| | EXP1427 | EXP1427 | | | |
|---|---|---|---|---|---|
| | API | C10 | | | |
| Time | Tₜ | Rₜ | Rₜ − Tₜ | | |
| 5 | 14.0 | 23.9 | 9.9 | | |
| 10 | 23.0 | 40.3 | 17.3 | | |
| 20 | 43.4 | 66.1 | 22.7 | | |
| 30 | 64.1 | 80.4 | 16.3 | | |
| 45 | 80.2 | 91.4 | 11.2 | | |
| 60 | 90.0 | 94.2 | 4.2 | | |
| 120 | 99.5 | 97.0 | 2.5 | | |
| | | | | SUM(Rₜ − Tₜ) | 77.4 |
| | F1: | 25.6 | | SUM(Rₜ) | 302.1 |
| | F2: | 39.6 | | SUM(Rₜ − | 0.256206554 |
| | | | | Tₜ)/SUM(Rₜ) | |
| | | | | F1 = | 25.62065541 |

TABLE 20
F2 analysis of C10 and zoledronic acid for EXP 1427

| | EXP1427 | | | |
|---|---|---|---|---|
| | API | EXP1427 C10 | | |
| Time | Tₜ | Rₜ | Rₜ − Tₜ | (Rₜ − Tₜ)² |
| 5 | 14.0 | 23.9 | 9.9 | 98.01 |
| 10 | 23.0 | 40.3 | 17.3 | 299.29 |
| 20 | 43.4 | 66.1 | 22.7 | 515.29 |
| 30 | 64.1 | 80.4 | 16.3 | 265.69 |
| 45 | 80.2 | 91.4 | 11.2 | 125.44 |
| 60 | 90.0 | 94.2 | 4.2 | 17.64 |
| 120 | 99.5 | 97.0 | 2.5 | 6.25 |
| | | | SUM(Rₜ − Tₜ)² | 1303.72 |
| | | | N = | 5 |
| | | | 1/N [SUM(Rₜ − Tₜ)²] | 260.744 |
| | | | 1 + 1/N [SUM(Rₜ − Tₜ)²] | 261.744 |
| | | | {1 + 1/N [SUM(Rₜ − Tₜ)²]}^{-0.5} | 0.061810411 |
| | | | {1 + 1/N [SUM(Rₜ − Tₜ)²]}^{-0.5} * 100 | 6.181041115 |
| | | | Log{{1 + 1/N [SUM(Rₜ − Tₜ)²]}^{-0.5} * 100} | 0.791061632 |
| | | | F2 (50 * Log{{1 + 1/N [SUM(Rₜ − Tₜ)²]}^{-0.5} * | 39.55308162 |
| | | | 100} | |

### (2) Tablets Including Alendronate, C10 and Sorbitol

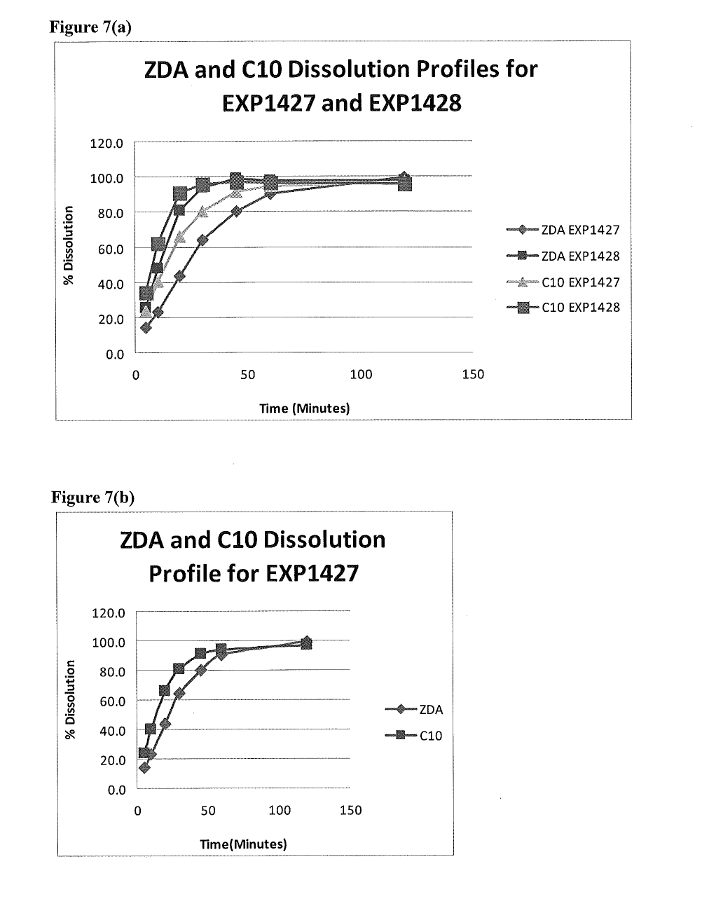

The tablets including alendronate, C10 and sorbitol have the same formulation as the tablets prepared using dry granulation and were prepared, according to similar procedures described above in Example 1(b). Dissolution rates were determined as in Example 2. The dissolution data and f1 and f2 analysis are provided in Tables 21-24. The dissolution profile and first derivative analysis are graphically described in FIGS. 8(a), 8(b) and 8(c). As shown in Tables 21-24 and FIGS. 8(a), 8(b) and 8(c), the f1 and f2 analysis demonstrate that the dissolution profile of alendronate and C10 is substantially similar.

TABLE 21
Dissolution profile of tablets including alendronate, C10 and sorbitol
DISSOLUTION RESULTS

| | | | | |
|---|---|---|---|---|
| Apparatus | RPM | 50 | Dissolution | 900 |
| | | | Volume | |
| Apparatus | RPM | | Dissolution | |
| | | | Volume | |

| | Time Points (minutes) | | |
|---|---|---|---|
| SODIUM | | | |
| ALENDRONATE | 10.0 | 20 | 30 |
| V1 | 74.5 | 98.5 | 98.6 |
| V2 | 64.7 | 88.4 | 88.7 |
| V3 | 71.9 | 92.3 | 94.5 |
| V4 | 67.5 | No Sample | 89.4 |
| V5 | 73.1 | 95.6 | 96.1 |
| V6 | 69.5 | 91.1 | 90.5 |
| mean | 70.2 | 93.2 | 93.0 |
| % RSD | 5.2 | 4.3 | 4.3 |

| | Time Points (minutes) | | | | |
|---|---|---|---|---|---|
| C10 | 5.0 | 10 | 15 | 20 | 30 |
| V1 | 40.3 | 71.3 | 89.1 | 96.4 | 96.1 |
| V2 | 39.2 | 71.2 | 87.3 | 94.7 | 95.4 |
| V3 | 39.1 | 70.4 | 87.2 | 93.6 | 95.0 |
| V4 | 44.3 | 74.4 | 91.0 | 95.4 | 93.9 |
| V5 | 41.0 | 72.8 | 90.6 | 96.7 | 95.5 |
| V6 | 39.7 | 66.6 | 86.9 | 91.4 | 90.3 |
| mean | 40.6 | 71.1 | 88.7 | 94.7 | 94.4 |
| % RSD | 4.8 | 3.7 | 2.0 | 2.1 | 2.2 |

TABLE 22
F1 analysis alendronate and C10

| | | Alen- | | | | | |
|---|---|---|---|---|---|---|---|
| | | dro- | | | | | |
| | % | nate | C10 | % | | | |
| Time | RSD | Tₜ | Rₜ | RSD | Rₜ − Tₜ | | |
| 5 | | | 40.6¹ | 4.8 | 40.6 | | |
| 10 | 5.2 | 70.2² | 71.1² | 3.7 | 0.9 | | |
| 20 | 4.3 | 93.2³ | 88.7³ | 2.0 | 4.5 | | |
| 30 | 4.3 | 93.0³ | 94.7³ | 2.1 | 1.7 | | |
| 45 | | | 94.4¹ | 2.2 | 94.4 | | |
| 60 | | | | | 0.0 | | |
| 120 | | | | | 0.0 | SUM(Rₜ − Tₜ) | 7.1 |
| | | F1: | 2.8 | | | SUM(Rₜ) | 254.5 |
| | | F2: | 76.2 | | | SUM(Rₜ − Tₜ)/ | 0.027898 |
| | | | | | | SUM(Rₜ) | |
| | | | | | | F1 = | 2.789784 |

¹Data graphed but does not meet criteria for F1-test and F2-test.
²Data meeting F1-test and F2-test and criteria
³Two points above 85%

TABLE 23
F2 analysis of alendronate and C10
F-2 ANALYSIS BN 06 07 02 T = 0

| | | Alendronate | C10 | | | |
|---|---|---|---|---|---|---|
| Time | % RSD | Tₜ | Rₜ | % RSD | Rₜ − Tₜ | (Rₜ − Tₜ)² |
| 5 | n/a | 0.0¹ | 40.6¹ | 4.8 | 40.6 | 1648.36 |
| 10 | 5.2 | 70.2² | 71.1² | 3.7 | 0.9 | 0.81 |
| 20 | 4.3 | 93.2³ | 88.7³ | 2.0 | −4.5 | 20.25 |
| 30 | 4.3 | 93.0³ | 94.7³ | 2.1 | 1.7 | 2.89 |
| 45 | | | 94.4¹ | 2.2 | 94.4 | 8911.36 |
| 60 | | | | | 0.0 | 0 |
| 120 | | | | | 0.0 | 0 |
| | | | | | SUM(Rₜ − Tₜ)² | 23.95 |
| | | | | | N= | 3 |
| | | | | | 1/N [SUM(Rₜ − Tₜ)²] | 7.983333333 |
| | | | | | 1 + 1/N [SUM(Rₜ − Tₜ)²] | 8.983333333 |
| | | | | | {1 + 1/N [SUM(Rₜ − | 0.333642405 |
| | | | | | Tₜ)²]} ^{-0.5} | |
| | | | | | {1 + 1/N [SUM(Rₜ − | 33.36424046 |
| | | | | | Tt)²]} ^{-0.5} * 100 | |
| | | | | | Log{{1 + 1/N | 1.523281243 |
| | | | | | [SUM(Rₜ −Tₜ)²]}^{-0.5} * 100 | |
| | | | | | F2 (50 * Log{{1 + 1/N | 76.16406213 |
| | | | | | [SUM(Rₜ −Tₜ)²]}^{-0.5} * 100 | |

¹Data graphed but does not meet criteria for F1-test and F2-test.
²Data meeting F1-test and F2-test and criteria
³Two points above 85%

TABLE 24
Delta analysis

| | | delta d/ | delta d/ |
|---|---|---|---|
| Time | delta t | delta t Alen | delta t C10 |
| 5 | 5 | | 8.12 |
| 10 | 5 | 7.02 | 6.10 |
| 20 | 10 | 2.30 | 1.76 |
| 30 | 10 | −0.02 | 0.60 |
| 45 | 15 | | −0.02 |
| 60 | 15 | | |
| 120 | 60 | | |

### (3) Tablets Including Acyline, C10 and Sorbitol

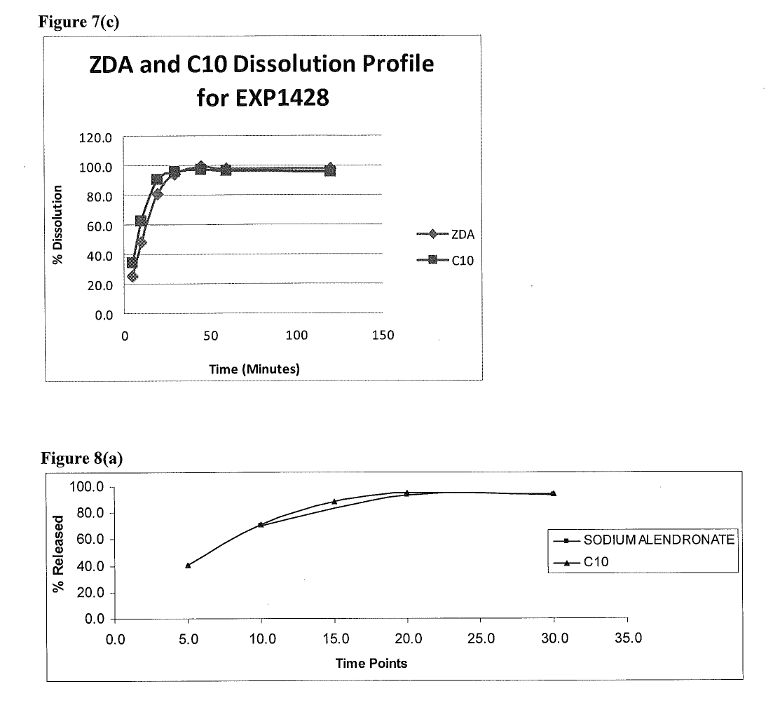

The tablets including acyline, C10 and sorbitol were similarly prepared as the tablets including zoledronic acid, C10 and sorbitol described above. The dissolution data and f1 and f2 analysis are provided in Tables 25-27. The dissolution profile and first derivative analysis are also graphically described in FIGS. 9(a) and 9(b). As shown in Tables 25-27 and FIGS. 9(a) and 9(b), the f1 and f2 analysis demonstrate that the dissolution profile of acyline and C10 is substantially similar.

TABLE 25
F1 analysis of tablets including acyline, C10 and sorbitol

| | | Ac- | | | | | |
|---|---|---|---|---|---|---|---|
| | % | yline | C10 | % | | | |
| Time | RSD | Tₜ | Rₜ | RSD | Rₜ − Tₜ | | |
| 5 | n/a | 0.0¹ | 0.0¹ | n/a | 0.0 | | |
| 10 | 46.4 | 8.9² | 4.7² | 155.7 | 4.2 | | |
| 20 | 14.2 | 55.5³ | 59.1³ | 13.9 | 3.6 | | |
| 30 | 5.5 | 89.3³ | 95.7³ | 5.5 | 6.4 | | |
| 45 | 1.6 | 100.0¹ | 103.8¹ | 0.1 | 3.8 | | |
| 60 | | | | | 0.0 | | |
| 120 | | | | | 0.0 | SUM(Rₜ − | 14.2 |
| | | | | | | Tₜ) | |
| | | F1: | 8.9 | | | SUM(Rₜ) | 159.5 |
| | | F2: | 65.1 | | | SUM(Rₜ − | 0.089028 |
| | | | | | | Tₜ)/ | |
| | | | | | | SUM(Rₜ) | |
| | | | | | | F1 = | 8.902821 |

¹Data used to calculate statistics but does not meet % RSD requirement for F1-test and F2-test.
²Data meeting F1-test and F2-test and criteria
³Data graphed but does not meet criteria for F1-test and F2-test.

TABLE 26
F2 analysis of tablets including acyline, C10 and sorbitol

| | | Acyline | C10 | | | |
|---|---|---|---|---|---|---|
| Time | % RSD | Tₜ | Rₜ | % RSD | Rₜ − Tₜ | (Rₜ − Tₜ)² |
| 5 | n/a | 0.0¹ | 0.0¹ | n/a | 0.0 | 0 |
| 10 | 46.4 | 8.9² | 4.7² | 155.7 | −4.2 | 17.64 |
| 20 | 14.2 | 55.5³ | 59.1³ | 13.9 | 3.6 | 12.96 |
| 30 | 5.5 | 89.3³ | 95.7³ | 5.5 | 6.4 | 40.96 |
| 45 | 1.6 | 100.0¹ | 103.8¹ | 0.1 | 3.8 | 14.44 |
| 60 | | | | | 0.0 | 0 |
| 120 | | | | | 0.0 | 0 |
| | | | | | SUM(Rₜ − Tₜ)² | 71.56 |
| | | | | | N= | 3 |
| | | | | | 1/N [SUM(Rₜ − Tₜ)²] | 23.85333333 |
| | | | | | 1 + 1/N [SUM(Rₜ − Tₜ)²] | 24.85333333 |
| | | | | | {1 + 1/N [SUM(Rₜ − | 0.200589261 |
| | | | | | Tₜ)²]} ^{-0.5} | |
| | | | | | {1 + 1/N [SUM(Rₜ − | 20.05892607 |
| | | | | | Tₜ)²]} ^{-0.5} * 100 | |
| | | | | | Log{{1 + 1/N | 1.302307678 |
| | | | | | [SUM(Rₜ −Tₜ)²]} ^{-0.5} * 100 | |
| | | | | | F2 (50 * Log{{1 + 1/N | 65.11538388 |
| | | | | | [SUM(Rₜ −Tₜ)²]} ^{-0.5} * 100 | |

¹Data used to calculate statistics butdoes not meet % RSD requirement for F1-test and F2-test.
²Data meeting F1-test and F2-test and criteria
³Data graphed but does not meet criteria for F1-test and F2-test.

TABLE 27
The first derivative analysis of tablets including acyline, C10 and sorbitol

| | | delta d/ | delta d/ |
|---|---|---|---|
| Time | delta t | delta t Acy | delta t C10 |
| 5 | 5 | 0.00 | 0.00 |
| 10 | 5 | 1.78 | 0.94 |
| 20 | 10 | 4.66 | 5.44 |
| 30 | 10 | 3.38 | 3.66 |
| 45 | 15 | 0.71 | 0.54 |
| 60 | 15 | | |
| 120 | 60 | | |

### Example 6

### Bioavailability Study for Different Administration Conditions

A human intubation study was conducted to evaluate the effect of different doses of the sodium salt of a medium chain fatty acid, capric acid (C10) on the absorption of low molecular weight heparin (LMWH) administered into the jejunum via nasojejunum intubation. All intrajejunal doses were applied through a custom made nasojejunal catheter which was placed in the jejunum on each dosing occasion As shown in Table 28 below, when a solution of parnaparin and the enhancer (C10) is administered (concurrent administration) (Entry 2), the bioavailability is improved compared to administering C10 15 minutes early then administering parnaparin (Entry 6). Since the drug and the enhancer are in solution together, this experiment replicates rapid and complete co-release of the drug and enhancer together from an enteric coated tablet in the gastrointestinal tract. These data emphasize the importance of having the active ingredient and enhancer release from the dosage from at substantially the same rate.

TABLE 28
The comparison data of bioavailability for parnaparin and
the enhancer (C10) under different administration conditions

| | | | | Mean | |
|---|---|---|---|---|---|
| | | | | Rel Bio | |
| | | Drug | | vs SC | |
| | | (Parnaparin) | C10 | LMWH and | Administration |
| Entry | Route | IU/tab | g/tab | C10 | conditions |
| 1 | intrajejunal | 20000 | 0.55 | 5.14 | bolus ij |
| | | | | | coadmin |
| 2 | intrajejunal | 20000 | 1.1 | 6.51 | bolus ij |
| | | | | | coadmin |
| 3 | intrajejunal | 45000 | 0.55 | 7.99 | bolus ij |
| | | | | | coadmin |
| 4 | intrajejunal | 45000 | 1.1 | 6.15 | bolus ij |
| | | | | | coadmin |
| 5 | intrajejunal | 45000 | 1.65 | 8.93 | bolus ij |
| | | | | | coadmin |
| 6 | intrajejunal | 20000 | 1.1 | 4.85 | C10 15 min |
| | | | | | before LMWH |

### Example 7

### Dissolution Study for Tablets of Octreotide Acetate

A study for testing the dissolution rate of tablets including octreotide acetate, C10 and sorbitol was carried out. This study served two purposes. The first was to confirm the unexpected observations made above using controlled variation in the affecting parameters. The second was to confirm that the advantages of this invention apply to larger molecules including peptides as well as smaller conventional compounds. Three different formulations were included in the study: (1) fast co-release of octreotide acetate and C10; (2) non-co-release formulation (slower release of octreotide acetate and faster co-release of C10): and (3) slower co-release of octreotide acetate and C10. The three formulations, manufacturing procedures as well as the dissolution rate are provided below.

### (1) Fast Co-Release of Octreotide Acetate and C10

A. Formulation

The formulation for fast co-release of octreotide acetate and C10 is provided in Table 29.

TABLE 29
Formulation for fast co-release of octreotide acetate and C10

| Ingredient Name | Mg/Tablet | Batch Size (g) |
|---|---|---|
| *Octreotide Acetate | 10.0 | 1.00 |
| Sodium Caprate | 550.0 | 55.00 |
| Parteck SI 150 | 136.5 | 13.65 |
| Stearic Acid | 3.5 | 0.35 |
| **Opadry II Yellow 85F32410 | 31.5 | 51.34 |
| **Acryl-EZE White 93018509 | 65.835 | 130.40 |

*Equivalent to 8.95 mg of Octreotide
**Includes overage for bulking cores

The octreotide acetate was removed from the freezer 1 hour before dispensing to allow the material equilibrate to room temperature.

B. Manufacturing

(i) Dispensing

All materials were dispensed into weight boats. Then the sodium caprate and octreotide acetate and Parteck SI 150 were screened through a 355 μm mesh into a stainless steel base pan. These materials were then transferred to a plastic container and blended together for 5 minutes. The stearic acid was then screened through a 355 μm mesh and added to the blended materials and blended for a further 2 minutes.

(ii) Tableting

The blended materials were then weighed out into lots of 700 mg and compressed at a PSI 4500 on a MTCM-I single punch tablet press fitted with a 16×8 mm oval shaped tool. The average hardness was 103 N and average weight was 699 mg. 60 tablets were compressed in total. These tablets were placed into Duma bottles and stored in the freezer over night. The tablets were removed from the freezer and allowed to equilibrate to room temperature

(iii) Film Coating (Sub)

51.34 g of Opadry I1 yellow 85F32410 and 205.26 ml of purified water was dispensed and mixed together for 40 minutes at high speed using a IKA stirrer. After the 40 minutes the solution was screened through a 90 μm mesh. Both the bulking Placebo cores and Octreotide Acetate tablets were placed into the O'Hara Labcoat M and the tablets were coated with the weight gain of approximately 4.5% weight gain using the following parameters.

Subcoat Filmcoating Parameters

Pan speed (10 rpm)

Supply air flow volume (100 m³/hr)

Supply air flow temperature (50° C.)

Exhaust air flow temperature (27.8° C.)

Atomisation pressure (0.6 Bar)

Solution spray speed (51 mL/min)

The tablets were dried for 10 minute in the pan at the end of the spraying process. 4.0% weight gain was achieved. The sub coated tablets were then placed in a double bag and stored in the freezer over night.

(iv) Film Coating (Enteric)

The tablets were removed from the freezer and allowed to equilibrate to room temperature. 130.4 g of Acryl-EZE White 93018509 and 521.6 ml of purified water was dispensed and mixed together for 20 minutes at speed using a IKA stirrer. After the 20 minutes the solution was screened through a 90 μm mesh.

Both the bulking cores and octreotide acetate sub coated tablets were placed into the O'Hara Labcoat M and the tablets were coated with the weight gain of approximately 10% weight gain using the following parameters.

Enteric Coat Filmcoating Parameters

Pan speed (10 rpm)

Supply air flow volume (100 m³/hr)

Supply air flow temperature (53° C.)

Exhaust air flow temperature (30° C.)

Atomisation pressure (0.6 Bar)

Solution spray speed (6 mL/min)

The tablets were heated for 10 minutes before the solution was applied. Also the sub coated tablets were dried for 10 minutes in the pan at the end of the spraying process. 10% weight gain was achieved. The enteric coated tablets were then placed in a double bag and stored in the freezer overnight. 12 tablets were submitted to the laboratory for dissolution and assay testing. The remaining tablets were stored in a double bag in the freezer.

C. Dissolution Rate

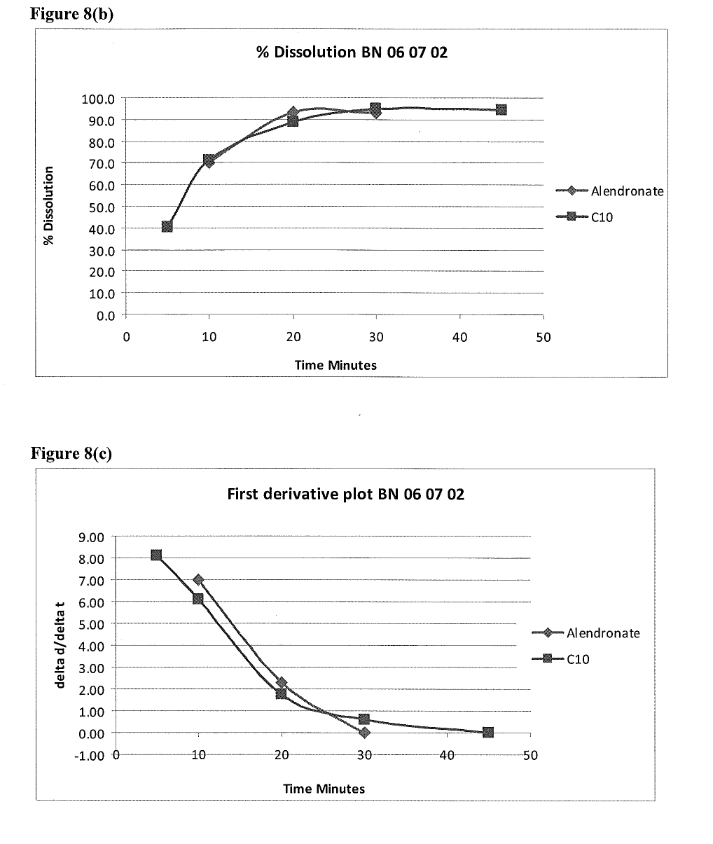

The dissolution rate of octreotide acetate and C10 are shown in Tables 30 and 31 respectively. The dissolution profile of octreotide acetate and C10 is graphically illustrated in FIG. 10.

As shown in FIG. 10 and Tables 30 and 31, the dissolution rate for the immediate co-release formulation of octreotide acetate and C10 is fast and significantly similar.

TABLE 30
Dissolution Rate of Octreotide Acetate

| | % Octreotide Dissolution | | | | | | | | | | |
|---|---|---|---|---|---|---|---|---|---|---|---|
| | | Buffer Stage Dissolution (minutes) | | | | | | | | | |
| Vessel | Acid | 10 | 15 | 30 | 45 | 60 | 120 | 240 | 480 | 720 | 1440 |
| 1 | 0 | 5.4 | 33.4 | 96.9 | 106.3 | 105.1 | 108.8 | 108.4 | 108.2 | 108.4 | 104.5 |
| 2 | 0 | 3.5 | 20.2 | 76.7 | 95.2 | 95.4 | 98.7 | 98.0 | 97.9 | 98.6 | 94.5 |
| 3 | 0 | 5.4 | 21.5 | 77.9 | 97.7 | 99.6 | 103.0 | 100.1 | 102.4 | 102.2 | 98.2 |
| 4 | 0 | 3.6 | 22.9 | 81.2 | 97.9 | 102.2 | 101.6 | 102.6 | 104.7 | 102.2 | 101.8 |
| 5 | 0 | 3.7 | 17.0 | 72.8 | 96.2 | 99.0 | 101.3 | 99.7 | 98.0 | 101.1 | 98.5 |
| 6 | 0 | 2.0 | 11.4 | 60.0 | 83.8 | 87.0 | 91.0 | 91.6 | 90.7 | 90.8 | 87.0 |
| Mean | 0 | 3.9 | 21.1 | 77.5 | 96.2 | 98.3 | 100.7 | 100.1 | 100.3 | 100.6 | 97.5 |
| % RSD | n/a | 1.4 | 7.0 | 12.5 | 7.5 | 6.8 | 6.1 | 5.7 | 6.4 | 6.0 | 6.4 |

*Dissolution Values Corrected using assay value as above.

TABLE 31
Dissolution Rate of C10

| | % C10 Dissolution | | | | | | | | | | |
|---|---|---|---|---|---|---|---|---|---|---|---|
| | | Buffer Stage Dissolution (minutes) | | | | | | | | | |
| Vessel | Acid | 10 | 15 | 30 | 45 | 60 | 120 | 240 | 480 | 720 | 1440 |
| 1 | 0 | 7.3 | 36.2 | 90.7 | 98.7 | 97.2 | 99.0 | 98.3 | 99.1 | 99.4 | 99.8 |
| 2 | 0 | 6.1 | 24.2 | 78.5 | 93.9 | 96.3 | 96.8 | 95.7 | 95.1 | 96.2 | 96.7 |
| 3 | 0 | 8.2 | 24.0 | 77.1 | 94.2 | 95.1 | 96.6 | 97.9 | 95.9 | 97.1 | 95.1 |
| 4 | 0 | 5.5 | 25.2 | 77.6 | 96.3 | 96.4 | 97.8 | 98.1 | 96.9 | 98.6 | 97.6 |
| 5 | 0 | 6.2 | 21.1 | 73.5 | 94.2 | 96.9 | 98.6 | 97.7 | 97.9 | 97.0 | 98.2 |
| 6 | 0 | 4.0 | 20.0 | 70.3 | 92.8 | 96.5 | 99.0 | 98.7 | 97.8 | 98.4 | 99.3 |
| Mean | 0 | 6.2 | 25.1 | 77.9 | 95.0 | 96.4 | 98.0 | 97.7 | 97.1 | 97.8 | 97.8 |
| % RSD | n/a | 23.1 | 23.1 | 8.9 | 2.3 | 0.8 | 1.1 | 1.1 | 1.5 | 1.2 | 1.8 |

### (2) Non-Co-Release Formulation (Slower Release of Octreotide Acetate and Faster Release of C10)

A. Formulation

The formulation of non-co-release of octreotide acetate and C10 is provided in Table 32.

TABLE 32
Non-co-release Formulation

| Ingredient Name | Mg/Tablet | Batch Size (g) |
|---|---|---|
| *Octreotide Acetate | 10.0 | 0.60 |
| Sodium Caprate | 550.0 | 33.00 |
| Methocel K4M | 136.5 | 8.19 |
| Stearic Acid | 3.5 | 0.21 |
| **Opadry II Yellow 85F32410 | 31.5 | 81.00 |
| **Acryl-EZE White 93018509 | 65.835 | 130.40 |

*Equivalent to 8.95 mg of Octreotide
**Includes overage for bulking cores

The octreotide acetate was removed from the freezer 1 hour before dispensing to allow the material equilibrate to room temperature.

B. Manufacturing

(i) Dispensing/Blending (a)

All materials above were dispensed into weight boats. Then the sodium caprate and stearic acid were screened through a 355 μm mesh into a stainless steel base pan. These materials were then transferred to a plastic container and blended together for 5 minutes.

(ii) Blending (b)

The octreotide Acetate and methocel K4M were also screened through a 355 μm mesh into a stainless steel base pan. These materials were then transferred to a plastic container and blended together for 5 minutes.

(iii) Tableting

Blended (a) material was taken and weighed out into portions consisting of 553.5 mg and slightly compressed at a force of 80 psi on a MTCM-1 single punch tablet press fitted with a 16×8 mm oval shaped tool. Then blend (b) was weighed into portions consisting of 146.5 mg and added on top of the slightly compressed tablet and these sections were fully compressed at a force of 4500 psi, producing a bi-layer tablet with an average hardness of 100 N and average weight of 700 mg. 58 tablets were compressed in total.

These tablets were placed into Duma bottles and stored in the freezer over night. The tablets were removed from the freezer and allowed to equilibrate to room temperature.

(iv) Film Coating (Sub)

81.0 g of Opadry I1 yellow 85F32410 and 324 ml of purified water was dispensed and mixed together for 40 minutes at high speed using a IKA stirrer. After the 40 minutes the solution was screened through a 90 μm mesh.

Both the bulking placebo cores and octreotide acetate tablets were placed into the O'Hara Labcoat M and the tablets were coated with the weight gain of approximately 4.5% weight gain using the following parameters.

Subcoat Film Coating Parameters

Pan speed (5-15 rpm)

Supply air flow volume (40 m³/hr)

Supply air flow temperature (50° C.)

Exhaust air flow temperature (27.6-29.3° C.)

Atomisation pressure (0.6 Bar)

Solution spray speed (5 mL/min)

The tablets were dried for 10 minute in the pan at the end of the spraying process. 4.5% weight gain was achieved. The sub coated tablets were then placed in a double bag and stored in the freezer over night.

(v) Film Coating (Enteric)

The tablets were removed from the freezer and allowed to equilibrate to room temperature.

130.4 g of Acryl-EZE White 9301 8509 and 521.6 ml of purified water was dispensed and mixed together for 20 minutes at speed using a IKA stirrer. After the 20 minutes the solution was screened through a 90 μm mesh. Both the bulking cores and octreotide acetate sub-coated tablets were placed into the O'Hara Labcoat M and the tablets were coated with the weight gain of approximately 10% weight gain using the following parameters.

Enteric Coat Film Coating Parameters

Pan speed (12 rpm)

Supply air flow volume (40 m³/hr)

Supply air flow temperature (50° C.)

Exhaust air flow temperature (28.3-31.4° C.)

Atomisation pressure (0.6 Bar)

Solution spray speed (6 mL/min)

The tablets were heated for 10 minutes before the solution was applied. Also the sub coated tablets were dried for 10 minute in the pan at the end of the spraying process. 10.3% weight gain was achieved. The enteric coated tablets were then placed in a double bag and stored in the freezer over night. 12 tablets were submitted to the laboratory for dissolution and assay testing. The remaining tablets were stored in a double bag in the freezer.

C. Dissolution Rate

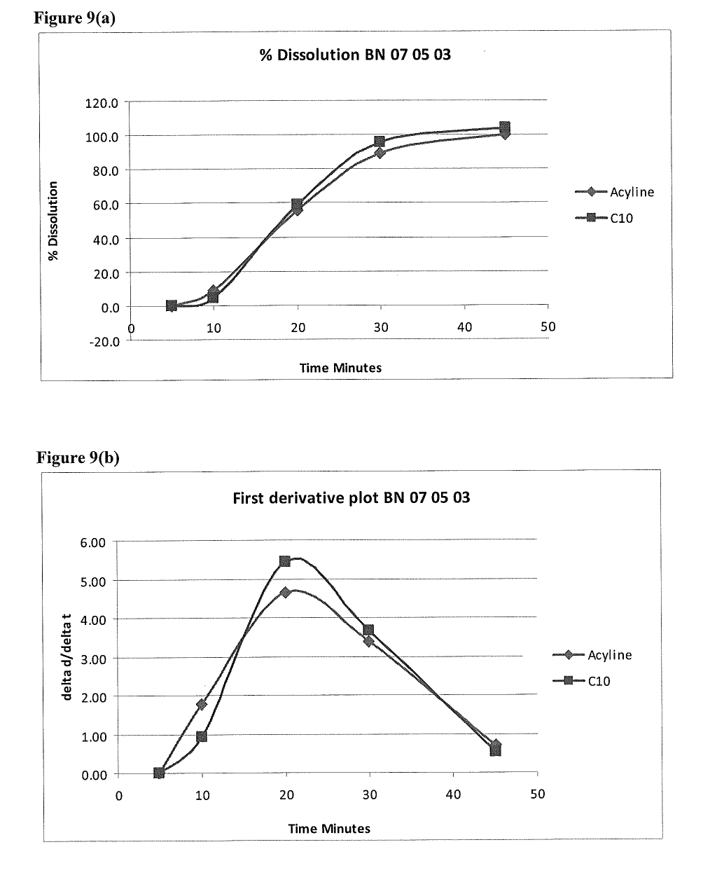

The dissolution rate of octreotide acetate and C10 are shown in Tables 33 and 34 respectively. The dissolution profile of octreotide acetate and C10 is graphically illustrated in FIG. 11.

As shown in FIG. 11 and Tables 33 and 34, the dissolution rate of C10 is instant and fast and the dissolution rate of octreotide acetate is slow.

TABLE 33
Dissolution Rate of Octreotide Acetate
Octreotide Dissolution % Dissolved *

| | | Buffer Stage (minutes) | | | | | | | | | | |
|---|---|---|---|---|---|---|---|---|---|---|---|---|
| Vessel | Acid | 10 | 15 | 30 | 45 | 60 | 120 | 240 | 40 | 720 | 1440 | Infinity |
| 1 | ND | 0.0 | 0.0 | 1.8 | 2.3 | 3.5 | 9.1 | 21.6 | 43.5 | 59.6 | 84.3 | 99.3 |
| 2 | ND | 0.0 | 0.0 | 0.0 | 2.4 | 4.5 | 12.7 | 26.5 | 51.2 | 67.1 | 96.7 | 102.6 |
| 3 | ND | 0.0 | 0.0 | 0.9 | 3.5 | 5.4 | 11.9 | 24.5 | 48.8 | 66.3 | 95.8 | 100.7 |
| 4 | ND | 0.0 | 0.0 | 1.0 | 2.5 | 4.3 | 10.7 | 23.6 | 41.4 | 57.5 | 91.8 | 98.9 |
| 5 | 2.6 | 0.0 | 0.0 | 1.6 | 3.1 | 5.2 | 12.6 | 26.8 | 47.7 | 65.7 | 100.3 | 102.0 |
| 6 | ND | 0.0 | 0.0 | 0.8 | 2.1 | 4.4 | 11.2 | 25.3 | 48.2 | 65.6 | 95.1 | 102.3 |
| Mean | 0.4 | 0.0 | 0.0 | 1.0 | 2.7 | 4.5 | 11.4 | 24.7 | 46.8 | 63.6 | 94.0 | 101.0 |
| % RSD | 245.0 | 0.0 | 0.0 | 62.7 | 19.4 | 15.2 | 12.0 | 8.0 | 7.8 | 6.3 | 5.8 | 1.6 |

* % Octreotide dissolved is corrected for Assay value, the label claim is assumed to be 7.95 mg/tablet for the % Dissolution calculation.

TABLE 34
Dissolution Rate of C10
C10 Dissolution % Dissolved

| | | Buffer Stage (minutes) | | | | | | | | | |
|---|---|---|---|---|---|---|---|---|---|---|---|
| Vessel | Acid | 10 | 15 | 30 | 45 | 60 | 120 | 240 | 40 | 720 | 1440 |
| 1 | ND | 2.7 | 17.7 | 64.2 | 94.0 | 98.7 | 100.1 | 99.1 | 98.9 | 96.7 | 96.4 |
| 2 | ND | 0.0 | 8.5 | 56.7 | 91.2 | 100.0 | 99.4 | 100.0 | 97.9 | 98.5 | 98.5 |
| 3 | ND | 0.0 | 2.1 | 25.1 | 60.1 | 86.8 | 96.4 | 96.7 | 96.4 | 95.6 | 96.6 |
| 4 | ND | 2.3 | 13.7 | 61.0 | 90.7 | 98.9 | 97.3 | 97.5 | 94.7 | 96.1 | 96.6 |
| 5 | ND | 4.3 | 16.6 | 60.8 | 90.2 | 93.9 | 92.9 | 92.7 | 92.4 | 92.6 | 92.4 |
| 6 | ND | 0.0 | 6.8 | 40.1 | 73.5 | 95.7 | 97.9 | 98.6 | 97.6 | 96.7 | 97.5 |
| Mean | ND | 1.5 | 10.9 | 51.3 | 83.3 | 95.7 | 97.3 | 97.4 | 96.3 | 96.0 | 96.3 |
| % RSD | n/a | 117.2 | 55.9 | 30.1 | 16.2 | 5.1 | 2.6 | 2.7 | 2.5 | 2.0 | 2.2 |

ND = Not Detected
n/a = not applicable

### (3) Slow Co-Release of Octreotide Acetate and C10

A. Formulation

The formulation of slower co-release of octreotide acetate and C10 is provided in Table 35.

TABLE 35
Formulation of slower co-release of octreotide acetate and C10

| Ingredient Name | Mg/Tablet | Batch Size (g) |
|---|---|---|
| *Octreotide Acetate | 10.0 | 0.80 |
| Sodium Caprate | 550.0 | 44.00 |
| Methocel K4M | 136.5 | 10.92 |
| Stearic Acid | 3.5 | 0.28 |
| **Opadry II Yellow 85F32410 | 31.5 | 81.00 |
| **Acryl-EZE White 93018509 | 65.835 | 130.40 |

*Equivalent to 8.95 mg of Octreotide
**Includes overage for bulking cores

B. Manufacturing

(i) Dispending/Blending

All materials were dispensed into weight boats. Then the sodium caprate and octreotide acetate and methocel K4M were screened through a 355 μm mesh into a stainless steel base pan. These materials were then transferred to a plastic container and blended together for 5 minutes. The stearic acid was then screened through a 355 μm mesh and added to the blended materials and blended for a further 2 minutes.

(ii) Tableting

The blended materials were weighed out into lots of 700 mg and compressed at a psi 4500 on a MTCM-1 single punch tablet press fitted with a 16×8 mm oval shaped tool. The average hardness was 105 N and average weight was 700 mg. 80 tablets were compressed in total. These tablets were placed into Duma bottles and stored in the freezer over night. The tablets were removed from the freezer and allowed to equilibrate to room temperature.

(iii) Film Coating (Sub)

81.0 g of Opadry II yellow 85F32410 and 324.0 ml of purified water was dispensed and mixed together for 40 minutes at high speed using a IKA stirrer. After the 40 minutes the solution was screened through a 90 μm mesh.

Both the bulking placebo cores and octreotide acetate tablets were placed into the O'Hara Labcoat M and the tablets were coated with the weight gain of approximately 4.5% weight gain using the following parameters.

Subcoat Filmcoating Parameters

Pan speed (10 rpm)

Supply air flow volume (100 m³/hr)

Supply air flow temperature (50° C.)

Exhaust air flow temperature (27.8° C.)

Atomisation pressure (0.6 Bar)

Solution spray speed (5 mL/min)

The tablets were dried for 10 minute in the pan at the end of the spraying process. 4.4% weight gain was achieved. The sub coated tablets were then placed in a double bag and stored in the freezer over night.

(iv) Film Coating (Enteric)

The tablets were removed from the freezer and allowed to equilibrate to room temperature. 130.4 g of Acryl-EZE White 93018509 and 521.6 ml of purified water was dispensed and mixed together for 20 minutes at speed using a IKA stirrer. After the 20 minutes the solution was screened through a 90 μm mesh.

Both the bulking cores and octreotide acetate sub coated tablets were placed into the O'Hara Labcoat M and the tablets were coated with the weight gain of approximately 10% weight gain using the following parameters.

Enteric Coat Filmcoating Parameters

Pan speed (10 rpm)

Supply air flow volume (100 m³/hr)

Supply air flow temperature (53° C.)

Exhaust air flow temperature (30° C.)

Atomisation pressure (0.6 Bar)

Solution spray speed (6 mL/min)

The tablets were heated for 10 minutes before the solution was applied. Also the sub coated tablets were dried for 10 minutes in the pan at the end of the spraying process. 9.6% weight gain was achieved. The enteric coated tablets were then placed in a double bag and stored in the freezer overnight. 12 tablets were submitted to the laboratory for dissolution and assay testing. The remaining tablets were stored in a double bag in the freezer.

C. Dissolution Rate

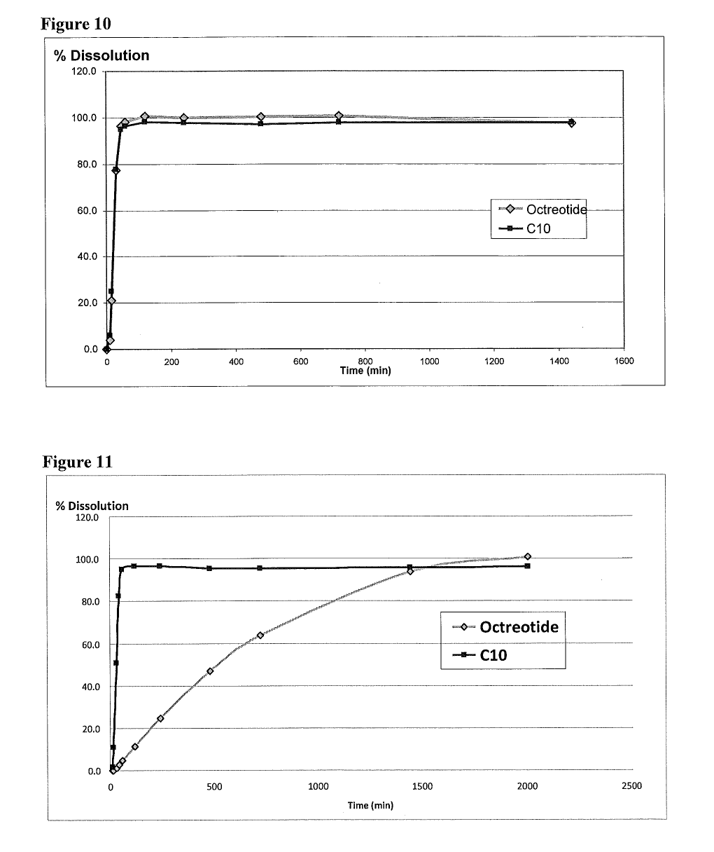

The dissolution rate of octreotide acetate and C10 is shown in Tables 39 and 40 respectively. The dissolution profile of octreotide acetate and C10 is graphically illustrated in FIG. 12.

As shown in FIG. 12 and Tables 36 and 37, the dissolution rate of octreotide acetate and C10 are significantly similar and are both slow.

TABLE 36
Dissolution Rate of Octreotide Acetate
Octreotide Dissolution % Dissolved

| | | Buffer Stage (minutes) | | | | | | | | | | |
|---|---|---|---|---|---|---|---|---|---|---|---|---|
| Vessel | Acid | 10 | 15 | 30 | 45 | 60 | 120 | 240 | 480 | 720 | 1440 | Infinity |
| 1 | ND | 0.0 | 0.0 | 1.2 | 2.7 | 5.1 | 16.4 | 31.1 | 56.9 | 78.6 | 102.8 | 102.6 |
| 2 | ND | 0.0 | 0.0 | 1.3 | 1.7 | 4.9 | 10.0 | 21.9 | 49.1 | 74.2 | 97.6 | 97.9 |
| 3 | ND | 0.0 | 0.0 | 1.6 | 2.6 | 5.5 | 16.4 | 33.6 | 61.0 | 82.5 | 104.2 | 104.0 |
| 4 | ND | 0.0 | 0.0 | 1.8 | 3.4 | 5.8 | 15.0 | 31.4 | 57.9 | 79.0 | 101.1 | 102.0 |
| 5 | ND | 0.0 | 0.0 | 1.5 | 2.9 | 5.1 | 13.0 | 25.1 | 53.1 | 75.3 | 94.2 | 95.6 |
| 6 | ND | 0.0 | 0.0 | 1.0 | 2.6 | 5.3 | 15.3 | 33.4 | 57.3 | 81.0 | 104.9 | 104.7 |
| Mean | NA | 0.0 | 0.0 | 1.4 | 2.7 | 5.3 | 14.4 | 29.4 | 55.9 | 78.4 | 100.8 | 101.2 |
| % RSD | NA | NA | NA | 21.1 | 20.2 | 5.9 | 17.2 | 16.4 | 7.5 | 4.1 | 4.1 | 3.6 |

TABLE 37
Dissolution Rate of C10
C10 Dissolution % Dissolved

| | | Buffer Stage (minutes) | | | | | | | | | | |
|---|---|---|---|---|---|---|---|---|---|---|---|---|
| Vessel | Acid | 10 | 15 | 30 | 45 | 60 | 120 | 240 | 480 | 720 | 1440 | Infinity |
| 1 | ND | 0.0 | 0.0 | 2.2 | 5.0 | 8.2 | 20.7 | 37.4 | 62.0 | 84.0 | 101.0 | 103.3 |
| 2 | ND | 0.0 | 0.0 | 2.1 | 4.7 | 7.5 | 15.8 | 29.7 | 58.7 | 83.3 | 101.7 | 102.9 |
| 3 | ND | 0.0 | 0.0 | 2.5 | 5.6 | 9.0 | 21.7 | 38.4 | 67.8 | 94.6 | 101.5 | 103.4 |
| 4 | ND | 0.0 | 0.0 | 2.4 | 5.7 | 9.3 | 20.7 | 37.9 | 65.8 | 88.1 | 102.2 | 105.0 |
| 5 | ND | 0.0 | 0.0 | 2.8 | 6.3 | 8.7 | 19.9 | 35.5 | 65.1 | 86.2 | 102.1 | 102.8 |
| 6 | ND | 0.0 | 0.0 | 1.3 | 5.1 | 8.6 | 19.9 | 37.2 | 63.7 | 87.3 | 99.0 | 101.6 |
| Mean | N/A | 0.0 | 0.0 | 2.2 | 5.4 | 8.5 | 19.8 | 36.0 | 63.8 | 87.2 | 101.3 | 103.2 |
| % RSD | N/A | N/A | N/A | 23.8 | 10.8 | 7.5 | 10.3 | 9.0 | 5.0 | 4.6 | 1.2 | 1.1 |

ND = Not Detected
N/A = Not Applicable

### (4) Bioavailability of Three Different Formulations

The bioavailability of octreotide acetate with the three different formulations described above was tested on female beagle dogs.

The tablets of three formulations discussed above were administered in four phases to eight female beagle dogs. Phase 1 corresponds to an IV dosage, which is a reference dosage form for the other treatments. Phase 2 corresponds to the fast co-release formulation. Phase 3 corresponds to the non-co-release formulation. Phase 4 corresponds to the slow co-release formulation. Each dog received a single oral tablet dose of 10 mg with a wash out period of at least one week in between each phase. The IV control formulation was administered to the same dogs (n=8) at dose of 50 μg/dog. Blood was collected for analysis of plasma drug levels at the following time points: 0 (pre-dose), 15, 30, 45 minutes, and 1, 1.5, 2, 3, 4, 8, 12 and 24 hours following dose administration. Plasma octreotide concentrations were determined by LCMS/MS.

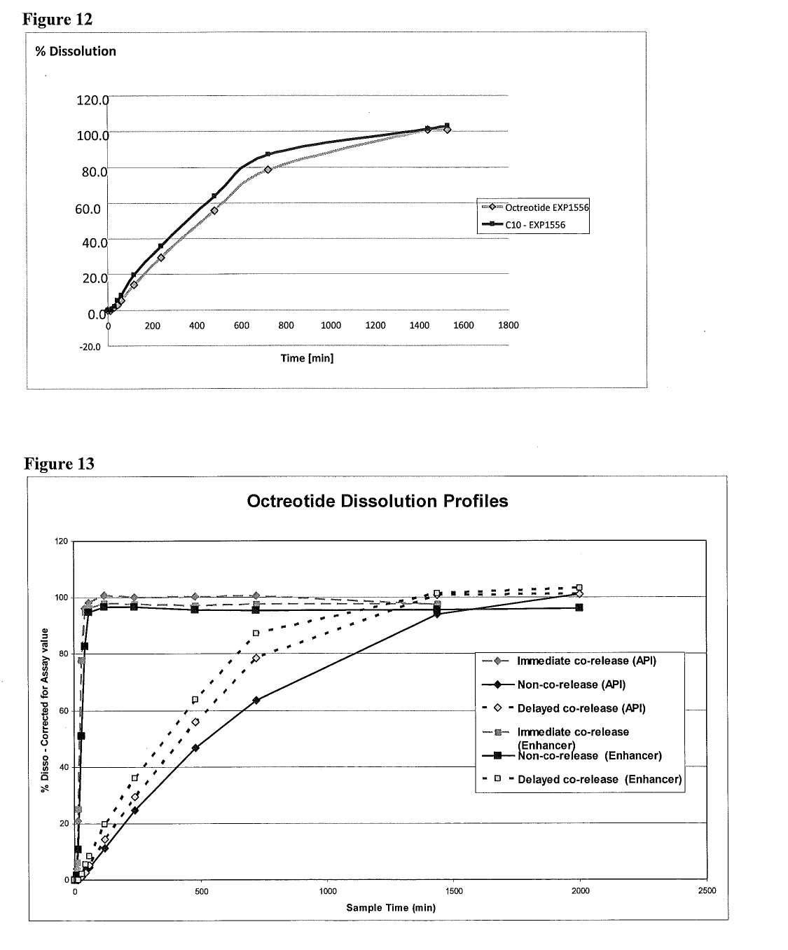

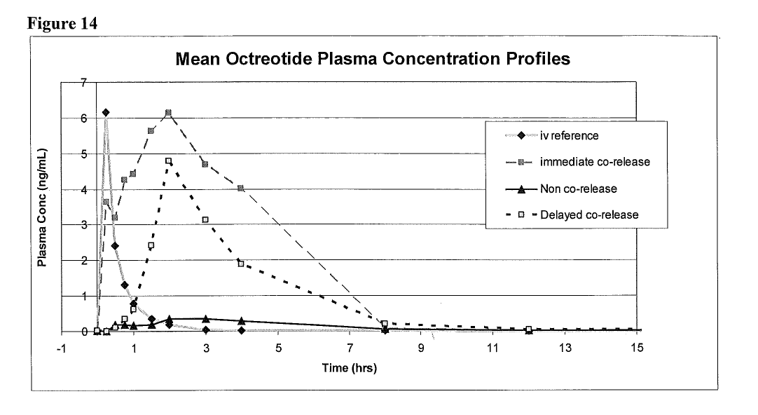

The pharmacokinetic parameters were calculated from the octreotide concentration-time data for each subject: Cₘₐₓ, T_{1/2}, AUC₍₀₋ₜ₎, and % bioavailability of the tested tablets relative to the intravenous injection (% F_{rel vs IV}). Pharmacokinetic parameters were calculated using macros written for MSExcel by Usansky et al. (See Joel I. Usansky, Ph.D., Atul Desai, M. S, and Diane Tsang-Liu, PH.D (1999), PK Functions for Microsoft Excel.) Group Mean, standard deviations, and % coefficient of variation(CV) values for all parameters were calculated using MSExcel calculation routines. The summary of the biological activity data (PK data) is provided in Tables 41 to 45. The comparison of the dissolution profiles is shown in FIG. 13. The comparison of plasma concentration profiles for phases 1-4 is shown in FIG. 14.

As shown in Tables 38-42 and FIG. 14, the bioavailability of the fast co-release formulation is highest among the three formulations. The bioavailability of the slow co-release formulation is lower than that of the fast co-release formulation but higher than IV and the non-co-release formulation. This study indicates that the fast co-release of octreotide and enhancer (fast co-release formulation) provides the greatest enhancement in Bioavailability % F_{rel vs iv}. The % bioavailability for the fast co-release formulation was 4.85% F_{rel vs iv} compared with 0.45% F_{rel vs iv} for the non-co-release formulation and 2.45% F_{rel vs iv} for the slow co-release formulation.

The f1 and f2 analyses are provided in Tables 42-47. As shown in Tables 42-47, the f1 and f2 analyses demonstrate that the dissolution profile of octreotide acetate and C10 is substantially similar.

TABLE 38
PK data for phase 1 (IV dose)

| TIME (hr) | F1 | F2 | F3 | F4 | F5 | F6 | F7 | F8 | Mean | SD |
|---|---|---|---|---|---|---|---|---|---|---|
| 0 | 0.00 | 0.00 | 0.00 | 0.00 | 0.00 | 0.00 | 0.04 | 0.00 | 0.00 | 0.01 |
| 0.25 | 5.88 | 10.40 | 5.63 | 6.33 | 4.70 | 6.04 | 4.71 | 5.66 | 6.17 | 1.81 |
| 0.50 | 2.30 | 3.86 | 2.46 | 2.68 | 1.65 | 2.20 | 1.80 | 2.42 | 2.42 | 0.67 |
| 0.75 | 1.18 | 2.13 | 1.33 | 1.68 | 0.92 | 1.10 | 0.89 | 1.30 | 1.32 | 0.41 |
| 1 | 0.63 | 1.46 | 0.84 | 0.94 | 0.63 | 0.60 | 0.46 | 0.75 | 0.79 | 0.31 |
| 1.5 | 0.28 | 0.67 | 0.38 | 0.43 | 0.31 | 0.26 | 0.31 | 0.18 | 0.35 | 0.15 |
| 2 | 0.16 | 0.38 | 0.21 | 0.19 | 0.09 | 0.12 | 0.13 | 0.15 | 0.18 | 0.09 |
| 3 | 0.06 | 0.12 | 0.06 | 0.07 | 0.01 | 0.03 | 0.00 | 0.00 | 0.04 | 0.04 |
| 4 | 0.03 | 0.07 | 0.03 | 0.03 | 0.00 | 0.04 | 0.00 | 0.00 | 0.02 | 0.02 |
| 8 | 0.01 | 0.00 | 0.10 | 0.00 | 0.01 | 0.04 | 0.00 | 0.00 | 0.02 | 0.03 |
| 12 | 0.00 | 0.00 | 0.00 | 0.02 | 0.00 | 0.00 | 0.00 | 0.00 | 0.00 | 0.01 |
| 24 | 0.00 | 0.00 | 0.00 | 0.00 | 0.05 | 0.01 | 0.00 | 0.00 | 0.00 | 0.00 |
| AUC | 3.02 | 5.55 | 3.57 | 3.68 | 2.28 | 3.14 | 2.28 | 2.83 | 3.34 | 1.00 |
| T½ | 0.93 | 0.54 | 1.40 | 0.50 | 0.31 | 1.18 | 0.36 | 0.28 | 0.69 | 0.43 |
| Cmax | 5.88 | 10.40 | 5.63 | 6.33 | 4.70 | 6.04 | 4.71 | 5.66 | 6.17 | 1.81 |
| Tmax | 0.25 | 0.25 | 0.25 | 0.25 | 0.25 | 0.25 | 0.25 | 0.25 | 0.25 | 0.00 |

TABLE 39
PK data for phase 2 (fast co-release formulation)

| TIME (hr) | F1 | F2 | F3 | F4 | F5 | F6 | F7 | F8 | Mean | SD |
|---|---|---|---|---|---|---|---|---|---|---|
| 0 | 0.00 | 0.00 | 0.00 | 0.00 | 0.00 | 0.00 | 0.00 | 0.00 | 0.00 | 0.00 |
| 0.25 | 0.01 | 19.70 | 0.00 | 0.00 | 0.00 | 0.00 | 9.45 | 0.00 | 3.65 | 7.28 |
| 0.50 | 0.00 | 14.10 | 6.47 | 0.00 | 2.94 | 0.00 | 1.93 | 0.13 | 3.20 | 4.95 |
| 0.75 | 0.00 | 8.82 | 10.10 | 0.00 | 8.24 | 5.21 | 0.90 | 0.81 | 4.26 | 4.33 |
| 1 | 0.00 | 5.11 | 11.50 | 0.00 | 11.10 | 4.87 | 0.41 | 2.45 | 4.43 | 4.70 |
| 1.5 | 0.00 | 1.63 | 14.50 | 0.00 | 13.10 | 1.37 | 0.13 | 14.30 | 5.63 | 6.94 |
| 2 | 0.00 | 0.84 | 13.20 | 0.00 | 15.00 | 2.31 | 0.03 | 17.70 | 6.13 | 7.72 |
| 3 | 0.11 | 0.39 | 10.70 | 0.52 | 5.40 | 0.65 | 0.00 | 19.70 | 4.68 | 7.14 |
| 4 | 0.89 | 0.19 | 1.98 | 23.40 | 1.63 | 0.29 | 0.00 | 3.76 | 4.02 | 7.93 |
| 8 | 0.11 | 0.00 | 0.16 | 0.17 | 0.10 | 0.00 | 0.00 | 0.10 | 0.08 | 0.07 |
| 12 | 0.03 | 0.00 | 0.02 | 0.08 | 0.02 | 0.00 | 0.00 | 0.00 | 0.02 | 0.03 |
| 24 | 0.00 | 0.00 | 0.00 | 0.04 | 0.00 | 0.25 | 0.00 | 0.00 | 0.04 | 0.09 |
| AUC | 2.98 | 14.87 | 42.04 | 60.57 | 34.84 | 8.44 | 3.31 | 51.11 | 27.27 | 22.76 |
| T½ | 1.55 | 0.54 | 1.03 | 2.71 | 1.09 | 0.79 | 0.22 | 0.71 | 1.08 | 0.77 |
| Cmax | 0.89 | 19.70 | 14.50 | 23.40 | 15.00 | 5.21 | 9.45 | 19.70 | 13.48 | 7.77 |
| Tmax | 4.00 | 0.25 | 1.50 | 4.00 | 2.00 | 0.75 | 0.25 | 3.00 | 1.97 | 1.56 |
| F % * | 0.57 | 1.54 | 6.78 | 9.48 | 7.61 | 1.55 | 0.84 | 10.41 | 4.85 | 4.14 |

TABLE 40
PK data for phase 3 (non-co-release formulation)

| TIME (hr) | F1 | F2 | F3 | F4 | F5 | F6 | F7 | F8 | Mean | SD |
|---|---|---|---|---|---|---|---|---|---|---|
| 0 | 0.00 | 0.00 | 0.12 | 0.00 | 0.01 | 0.08 | 0.00 | 0.00 | 0.03 | 0.05 |
| 0.25 | 0.00 | 0.00 | 0.00 | 0.03 | 0.00 | 0.00 | 0.00 | 0.00 | 0.00 | 0.01 |
| 0.50 | 0.00 | 0.37 | 0.00 | 0.24 | 0.00 | 0.02 | 0.08 | 0.74 | 0.18 | 0.26 |
| 0.75 | 0.00 | 0.34 | 0.03 | 0.55 | 0.00 | 0.06 | 0.14 | 0.44 | 0.19 | 0.22 |
| 1 | 0.03 | 0.10 | 0.00 | 0.66 | 0.08 | 0.00 | 0.17 | 0.24 | 0.16 | 0.22 |
| 1.5 | 0.05 | 0.00 | 0.01 | 0.64 | 0.30 | 0.00 | 0.36 | 0.11 | 0.18 | 0.23 |
| 2 | 0.03 | 0.00 | 0.03 | 0.70 | 0.71 | 0.78 | 0.42 | 0.14 | 0.35 | 0.34 |
| 3 | 0.15 | 0.00 | 0.24 | 0.35 | 0.38 | 1.41 | 0.13 | 0.09 | 0.34 | 0.45 |
| 4 | 0.10 | 0.00 | 0.28 | 0.47 | 0.47 | 0.36 | 0.56 | 0.05 | 0.28 | 0.21 |
| 8 | 0.01 | 0.00 | 0.20 | 0.21 | 0.06 | 0.00 | 0.03 | 0.00 | 0.06 | 0.09 |
| 12 | 0.00 | 0.02 | 0.04 | 0.04 | 0.02 | 0.00 | 0.00 | 0.00 | 0.01 | 0.02 |
| 24 | 0.00 | 0.00 | 0.04 | 0.05 | 0.00 | 0.05 | 0.00 | 0.20 | 0.04 | 0.07 |
| AUC | 0.47 | 0.34 | 2.35 | 4.25 | 2.70 | 3.25 | 2.28 | 1.94 | 2.20 | 1.31 |
| T½ | 1.15 | 0.26 | 7.41 | 5.50 | 2.00 | 0.51 | 1.78 | 1.00 | 2.45 | 2.59 |
| Cmax | 0.15 | 0.37 | 0.28 | 0.70 | 0.71 | 1.41 | 0.56 | 0.74 | 0.61 | 0.39 |
| Tmax | 3.00 | 0.50 | 4.00 | 2.00 | 2.00 | 3.00 | 4.00 | 0.50 | 2.38 | 1.38 |
| F % * | 0.10 | 0.04 | 0.41 | 0.73 | 0.64 | 0.65 | 0.63 | 0.43 | 0.45 | 0.26 |

TABLE 41
PK data for phase 4 (slow co-release formulation)

| TIME (hr) | F1 | F2 | F3 | F4 | F5 | F6 | F7 | F8 | Mean | SD |
|---|---|---|---|---|---|---|---|---|---|---|
| 0 | 0.05 | 0.00 | 0.00 | 0.00 | 0.00 | 0.00 | 0.07 | 0.00 | 0.01 | 0.03 |
| 0.25 | 0.00 | 0.00 | 0.00 | 0.00 | 0.00 | 0.00 | 0.00 | 0.00 | 0.00 | 0.00 |
| 0.50 | 0.00 | 0.19 | 0.00 | 0.00 | 0.00 | 0.61 | 0.00 | 0.11 | 0.11 | 0.21 |
| 0.75 | 0.00 | 0.59 | 0.00 | 0.00 | 0.00 | 1.54 | 0.25 | 0.41 | 0.35 | 0.53 |
| 1 | 0.00 | 1.16 | 0.00 | 0.00 | 0.64 | 1.83 | 0.75 | 0.47 | 0.61 | 0.65 |
| 1.5 | 0.00 | 6.18 | 0.00 | 0.00 | 2.56 | 3.79 | 3.37 | 3.37 | 2.41 | 2.25 |
| 2 | 0.00 | 19.60 | 0.00 | 0.00 | 6.23 | 5.77 | 2.46 | 4.22 | 4.79 | 6.51 |
| 3 | 0.00 | 9.65 | 0.97 | 0.00 | 6.15 | 1.78 | 1.60 | 4.83 | 3.12 | 3.44 |
| 4 | 0.32 | 3.85 | 0.25 | 0.13 | 2.40 | 1.85 | 2.51 | 3.73 | 1.88 | 1.52 |
| 8 | 0.02 | 0.15 | 1.07 | 0.11 | 0.18 | 0.02 | 0.04 | 0.03 | 0.20 | 0.36 |
| 12 | 0.00 | 0.17 | 0.13 | 0.07 | 0.00 | 0.00 | 0.00 | 0.00 | 0.05 | 0.07 |
| 24 | 0.00 | 0.07 | 0.00 | 0.00 | 0.00 | 0.00 | 0.00 | 0.00 | 0.01 | 0.02 |
| AUC | 0.89 | 40.05 | 6.90 | 1.33 | 19.06 | 13.92 | 11.90 | 19.44 | 14.19 | 12.65 |
| T½ | 1.02 | 2.80 | 5.20 | 8.49 | 1.10 | 0.71 | 1.01 | 0.66 | 2.62 | 2.83 |
| Cmax | 0.32 | 19.60 | 1.07 | 0.13 | 6.23 | 5.77 | 3.37 | 4.83 | 5.17 | 6.31 |
| Tmax | 4.00 | 2.00 | 8.00 | 4.00 | 2.00 | 2.00 | 1.50 | 3.00 | 3.31 | 2.12 |
| F %* | 0.17 | 4.21 | 1.13 | 0.21 | 4.22 | 2.59 | 3.05 | 4.01 | 2.45 | 1.73 |

TABLE 42
F1 analysis of fast co-release formulation of tablets including
octreotide acetate and C10

| Time | Tₜ | Rₜ | Rₜ − Tₜ | |
|---|---|---|---|---|
| 10 | 3.9 | 6.2 | 2.3 | |
| 15 | 21.1 | 25.1 | 4.0 | |
| 30 | 77.5 | 77.9 | 0.4 | |
| 45 | 96.2 | 95.0 | 1.2 | |
| 60 | 98.3 | 96.4 | 1.9 | |
| 120 | 100.7 | 98.0 | 2.7 | |
| 240 | 100.0 | 97.7 | 2.3 | |
| | | | | SUM(Rₜ − Tₜ) |
| | F1: | 3.9 | | SUM(Rₜ) |
| | F2: | 79.3 | | SUM(Rₜ − Tₜ)/SUM(Rₜ) |
| | | | | F1 = |

TABLE 43
F2 analysis of fast co-release formulation of tablets including
octreotide acetate and C10

| Time | Tₜ | Rₜ | Rₜ − Tₜ | (Rₜ − Tₜ)² |
|---|---|---|---|---|
| 45 | 3.9 | 6.2 | 2.3 | 5.29 |
| 60 | 21.1 | 25.1 | 4.0 | 16 |
| 120 | 77.5 | 77.9 | 0.4 | 0.16 |
| 240 | 96.2 | 95.0 | −1.2 | 1.44 |
| 480 | 98.3 | 96.4 | −1.9 | 3.61 |
| 720 | 100.7 | 98.0 | −2.7 | 7.29 |
| 1440 | 100.0 | 97.7 | −2.3 | 5.29 |
| | | | SUM(Rₜ − Tₜ)² | 22.89 |
| | | | N = | 4 |
| | | | 1/N [SUM(Rₜ − Tₜ)²] | 5.7225 |
| | | | 1 + 1/N [SUM(Rₜ − Tₜ)²] | 6.7225 |
| | | | {1 + 1/N [SUM(Rₜ − Tₜ)²]}^{-0.5} | 0.385686639 |
| | | | {1 + 1/N [SUM(Rₜ − Tₜ)²]}^{-0.5} * 100 | 38.56866393 |
| | | | Log{{1 + 1/N [SUM(Rₜ − Tₜ)²]}^{-0.5} * 100} | 1.586234595 |
| | | | F2 (50 * Log{{1 + 1/N [SUM(Rₜ − Tₜ)²]}^{-0.5} * 100} | 79.31172973 |

TABLE 44
F1 analysis of non-co-release formulation of tablets including
octreotide acetate and C10

| Time | Tₜ | Rₜ | Rₜ − Tₜ | | |
|---|---|---|---|---|---|
| 30 | 1.0 | 51.3 | 50.3 | | |
| 45 | 2.7 | 83.3 | 80.6 | | |
| 60 | 4.5 | 95.7 | 91.2 | | |
| 120 | 11.4 | 97.3 | 85.9 | | |
| 240 | 24.7 | 97.4 | 72.7 | | |
| 480 | 46.8 | 96.3 | 49.5 | | |
| 720 | 63.6 | 96.0 | 32.4 | | |
| | | | | SUM(Rₜ − Tₜ) | 379.9 |
| | F1: | 80.8 | | SUM(Rₜ) | 470.0 |
| | F2: | 5.6 | | SUM(Rₜ − Tₜ)/SUM(Rₜ) | 0.808297872 |
| | | | | F1 = | 80.82978723 |

TABLE 45
F2 analysis of non-co-release formulation of tablets including
octreotide acetate and C10

| Time | Tₜ | Rₜ | Rₜ − Tₜ | (Rₜ − Tₜ)² |
|---|---|---|---|---|
| 30 | 1.0 | 51.3 | 50.3 | 2530.09 |
| 45 | 2.7 | 83.3 | 80.6 | 6496.36 |
| 60 | 4.5 | 95.7 | 91.2 | 8317.44 |
| 120 | 11.4 | 97.3 | 85.9 | 7378.81 |
| 240 | 24.7 | 97.4 | 72.7 | 5285.29 |
| 480 | 46.8 | 96.3 | 49.5 | 2450.25 |
| 720 | 63.6 | 96.0 | 32.4 | 1049.76 |
| | | | SUM(Rₜ − Tₜ)² | 29928.15 |
| | | | N = | 5 |
| | | | 1/N [SUM(Rₜ − Tₜ)²] | 5985.63 |
| | | | 1 + 1/N [SUM(Rₜ − Tₜ)²] | 5986.63 |
| | | | {1 + 1/N [SUM(Rₜ − Tₜ)²]}^{-0.5} | 0.012924352 |
| | | | {1 + 1/N [SUM(Rₜ − Tₜ)²]}^{-0.5} * 100 | 1.29243524 |
| | | | Log{{1 + 1/N [SUM(Rₜ − Tₜ)²]}^{-0.5} * 100} | 0.111408791 |
| | | | F2 (50 * Log{{1 + 1/N [SUM(Rₜ − Tₜ)²]}^{-0.5} * 100} | 5.570439558 |

TABLE 46
F1 analysis of slow co-release formulation of tablets including
octreotide acetate and C10

| Time | Tₜ | Rₜ | Rₜ − Tₜ | | |
|---|---|---|---|---|---|
| 45 | 2.7 | 5.4 | 2.7 | | |
| 60 | 5.3 | 8.5 | 3.2 | | |
| 120 | 14.4 | 19.8 | 5.4 | | |
| 240 | 29.4 | 36.0 | 6.6 | | |
| 480 | 55.9 | 63.8 | 7.9 | | |
| 720 | 78.4 | 87.2 | 8.8 | | |
| 1440 | 100.8 | 101.3 | 0.5 | | |
| | | | | SUM(Rₜ − Tₜ) | 35.1 |
| | F1: | 10.9 | | SUM(Rₜ) | 322.0 |
| | F2: | 61.7 | | SUM(Rₜ − | 0.109006 |
| | | | | Tₜ)/SUM(Rₜ) | |
| | | | | F1 = | 10.90062 |

TABLE 47
F2 analysis of slow co-release formulation of tablets including
octreotide acetate and C10

| Time | Tₜ | Rₜ | Rₜ − Tₜ | (Rₜ − Tₜ)² |
|---|---|---|---|---|
| 45 | 2.7 | 5.4 | 2.7 | 7.29 |
| 60 | 5.3 | 8.5 | 3.2 | 10.24 |
| 120 | 14.4 | 19.8 | 5.4 | 29.16 |
| 240 | 29.4 | 36.0 | 6.6 | 43.56 |
| 480 | 55.9 | 63.8 | 7.9 | 62.41 |
| 720 | 78.4 | 87.2 | 8.8 | 77.44 |
| 1440 | 100.8 | 101.3 | 0.5 | 0.25 |
| | | | SUM(Rₜ − Tₜ)² | 230.35 |
| | | | N = | 7 |
| | | | 1/N [SUM(Rₜ − Tₜ)²] | 32.90714286 |
| | | | 1 + 1/N [SUM(Rₜ − Tₜ)²] | 33.90714286 |
| | | | {1 + 1/N [SUM(Rₜ − Tₜ)²]}^{-0.5} | 0.171733255 |
| | | | {1 + 1/N [SUM(Rₜ − Tₜ)²]}^{-0.5} * 100 | 17.17332552 |
| | | | Log{{1 + 1/N [SUM(Rₜ − Tₜ)²]}^{-0.5} * 100} | 1.234854402 |
| | | | F2 (50 * Log{{1 + 1/N [SUM(Rₜ − Tₜ)²]}^{-0.5} * 100} | 61.7427201 |

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A pharmaceutical composition, which is effective in providing therapeutically effective blood levels of a therapeutically active ingredient to a subject when administered to a gastrointestinal tract, comprising:
(i) a therapeutically effective amount of a therapeutically active ingredient;
(ii) at least one water soluble enhancer; and
(iii) a saccharide;
wherein the pharmaceutical composition provides rapid release of the therapeutically active ingredient and the enhancer after the pharmaceutical composition enters the intestine of a subject; and
wherein the pharmaceutical composition, in the form of a dosage form without coating, provides an in vitro dissolution of at least 80% of the therapeutically active ingredient and the enhancer in 20 minutes.

2. (canceled)

3. The pharmaceutical composition of claim 1, wherein the pharmaceutical composition, in the form of a dosage form without coating, provides an in vitro dissolution of at least 95% of the therapeutically active ingredient and/or the enhancer in 40 minutes.

4. (canceled)

5. The pharmaceutical composition of claim 1 wherein the dissolution is measured in 900 mL pH 6.8 phosphate buffer at 37° C. with a USP Paddle Apparatus at 50 rpm.

6. A pharmaceutical composition, which is effective in providing therapeutically effective blood levels of a therapeutically active ingredient to a subject when administered to a gastrointestinal tract, comprising:
(i) a therapeutically effective amount of a therapeutically active ingredient;
(ii) at least one water soluble enhancer; and
(iii) a saccharide;
wherein the pharmaceutical composition provides a substantially similar release rate of the therapeutically active ingredient and the enhancer after the pharmaceutical composition enters the intestine of a subject; and
wherein the substantially similar release rate is a ratio of the time for a percentage of the therapeutically active agent to be released in an in vitro dissolution from a dosage form of the pharmaceutical composition without coating to the time for the same percentage of the enhancer to be released of about 1.3 to about 0.7.

7. (canceled)

8. The pharmaceutical composition of claim 6, wherein the dissolution is measured in 900 mL pH 6.8 phosphate buffer at 37° C. with a USP Paddle Apparatus at 50 rpm.

9. The pharmaceutical composition of claim 6, wherein f1 for the dissolution profile of the enhancer and the therapeutically active ingredient is less than about 15.

10. The pharmaceutical composition of claim 6, wherein f2 for the dissolution profile of the enhancer and the therapeutically active ingredient is in a range of about 50 to about 100.

11. (canceled)

12. The pharmaceutical composition of claim 1, wherein the pharmaceutical composition when present in a dosage form without coating has a disintegration time in water of less than about 15 minutes at 37° C.

13. (canceled)

14. A method of providing a pharmaceutical composition for oral administration in a single dosage unit with a patient acceptable size, wherein the composition comprises:
(i) a therapeutically effective amount of a therapeutically active ingredient;
(ii) at least one water soluble enhancer; and
(iii) a saccharide;
the method comprising directly compressing or dry granulating the enhancer without adding any moisture agent before preparing the dosage form.

15. The method of claim 14, further comprising mixing the compressed or granulated enhancer with the therapeutically active ingredient and the saccharide.

16. The method of claim 14, wherein the enhancer is compressed or granulated by itself.

17. The method of claim 14, wherein the patient acceptable size is no more than about 1.2 gram/per dosage unit.

18. (canceled)

19. A method for the treatment and/or prevention of a medical condition, which is effective in providing therapeutically effective blood levels of a therapeutically active ingredient to a subject when administered to a gastrointestinal tract of the subject, the method comprising administering orally to the subject the pharmaceutical composition of claim 1.

20. The pharmaceutical composition of claim 1, wherein the saccharide is selected from the group consisting of sorbitol, mannitol, xylitol, sucrose, and a combination thereof.

21. (canceled)

22. The pharmaceutical composition of claim 1, wherein the weight ratio of the enhancer and saccharide is about 3:1 to 6:1.

23-24. (canceled)

25. The pharmaceutical composition of claim 1, wherein the therapeutically active ingredient is a bisphosphonate compound, low molecular weight heparin, or hydrophilic or macromolecular drug.

26-34. (canceled)

35. The pharmaceutical composition of claim 1, wherein the enhancer is a medium chain fatty acid or a salt, ester, ether, or derivative of a medium chain fatty acid and has a carbon chain length of from about 4 to about 20 carbon atoms.

36. (canceled)

37. The pharmaceutical composition of claim 1, wherein the enhancer is a sodium salt of a medium chain fatty acid.

38. The pharmaceutical composition of claim 1, wherein the enhancer is selected from the group consisting of sodium caprylate, sodium caprate and sodium laurate.

39. The pharmaceutical composition of claim 1, wherein the enhancer is sodium caprate.

40. The pharmaceutical composition of claim 1, wherein the enhancer is present in a weight percentage of at least about 50 percent of the total weight of the pharmaceutical composition in one dosage unit.

41. (canceled)

42. The pharmaceutical composition of claim 1, wherein the amount of enhancer is at least about 2.0 mmol in one dosage unit.

43-44. (canceled)

45. The pharmaceutical composition of claim 1, wherein the enhancer is compressed or granulated without adding any moisture agent before preparing the pharmaceutical composition.

46. The pharmaceutical composition or method of claim 45, wherein the enhancer is directly compressed before preparing the pharmaceutical composition.

47. The pharmaceutical composition or method of claim 45, wherein the enhancer is dry granulated before preparing the pharmaceutical composition.

48. The pharmaceutical composition of claim 1, wherein the composition is in a dosage form selected from the group consisting of a tablet, a particulate, a multi-particulate, a capsule, a pellet, an encapsulated pellet, and an encapsulated micro-particulate.

49. The pharmaceutical composition of claim 1, wherein the composition is further coated, compressed and/or packaged.

50. A solid oral dosage from comprising the pharmaceutical composition of claim 1.

51. The solid oral dosage form of claim 50, in a form selected from the group consisting of a tablet, a particulate, a multi-particulate, a capsule, a pellet, an encapsulated pellet, and an encapsulated micro-particulate.

52. The method of claim 19, wherein the medical condition is selected from the group consisting of osteoporosis, rheumatoid arthritis, bone fracture, excessive bone resorption, bone cancer, and a combination thereof.
